(19) United States
(12) Patent Application Publication
(10) Pub. No.: US 2009/0258435 A1
(43) Pub. Date: Oct. 15, 2009

(54) BIOTIN-RECEPTOR REAGENTS FOR SENSITIVITY MODULATION IN ASSAYS

(75) Inventors: Sandra A. Lewisch, Bear, DE (US); Pratap Singh, Wilmington, DE (US); Viral Desai, Newark, DE (US); Karen L. Krakowski, Chesapeake City, MD (US); James E. Duffy, Landenberg, PA (US)

Correspondence Address: DADE BEHRING INC.;LAW AND PATENTS, 1717 DEERFIELD ROAD, DEERFIELD, IL, 60015, US

(73) Assignee: SIEMENS HEALTHCARE DIAGNOSTICS INC., A CORPORATION OF CALIFORNIA, Deerfield, IL (US)

(21) Appl. No.: 12/103,245

(22) Filed: Apr. 15, 2008

(51) Int. Cl. G01N 33/566; G01N 33/536

(52) U.S. Cl. 436/501; 436/536

(57) ABSTRACT

Methods are disclosed for designing an antibody reagent for use in an assay for the detection of an analyte to obtain an optimum assay sensitivity and/or dynamic range. The antibody reagent is a conjugate of a small molecule attached by a spacer group to an antibody for the analyte. The method comprises controlling, in the preparation of the conjugate, reaction parameters comprising the hydrophobicity or hydrophilicity of the spacer group, the length of the spacer group, the number of molecules of the small molecule attached to the antibody and the point of attachment of the small molecule to the antibody to obtain an optimum assay sensitivity and/or dynamic range. In some embodiments the method comprises preparing two or more conjugates by selecting a set of parameters for each conjugate wherein the set of parameters is different for each conjugate, conducting an assay for the analyte employing each conjugate and selecting for use in the assay the conjugate that provides the optimum assay sensitivity and/or dynamic range.

### BACKGROUND

The present invention relates to biotin-receptor compounds and compositions that find use, for example, in assays for analytes, such as, e.g., immunoassays, receptor assays and nucleic acid assays. In particular, the present reagents permit modulation of sensitivity in such assays.

In the above assays it is often necessary to bind together two components, one being, for example, a specific binding member and the other being another assay component such as, for example, a receptor. Reagents containing biotin such as, for example, biotinylated antibodies, are convenient for use in such assays. Such biotin reagents generally have an antibody or antibody fragment conjugated to biotin. A biotin-binding reagent is also employed that has a moiety that binds biotin (biotin-binding moiety) such as, for example, avidin or streptavidin, bound to other components. To bring about binding of the two components, it is merely necessary to combine the biotin reagent with the avidin reagent. The binding interactions between biotin and the biotin-binding site of avidin are the result of, among others, formation of multiple hydrogen bonds and van der Waals interactions between biotin and avidin together with the ordering of surface polypeptide loops that bury the biotin in the protein interior.

There is a continuing need to develop fast and accurate diagnostic methods to measure levels of analytes in biological and other samples. In particular, there is a continuing need for improvement of biotinylated binding reagents for use in assays. Such reagents should provide for optimum performance including sensitivity.

### SUMMARY

One embodiment of the present invention is a method for designing an antibody reagent for use in an assay for the detection of an analyte to obtain optimum assay sensitivity. The antibody reagent is a conjugate of a small molecule attached by a spacer group to an antibody for the analyte. The method comprises controlling, in the preparation of the conjugate, reaction parameters comprising the hydrophobicity or hydrophilicity of the spacer group, the length of the spacer group, the number of molecules of the small molecule attached to the antibody and the point of attachment of the small molecule to the antibody to obtain an optimum assay sensitivity. In some embodiments the method comprises preparing two or more conjugates by selecting a set of parameters for each conjugate wherein the set of parameters is different for each conjugate, conducting an assay for the analyte employing each conjugate and selecting for use in the assay the conjugate that provides the optimum assay sensitivity.

Another embodiment of the present invention is a method for designing a biotinylated antibody reagent for use in an assay for the detection of an analyte to obtain an optimum assay sensitivity. The biotinylated antibody reagent is a conjugate of biotin attached by a spacer group to an antibody for the analyte. The method comprises controlling, in the preparation of the conjugate, reaction parameters comprising: (a) the hydrophobicity or hydrophilicity of the spacer group, (b) the length of the spacer group wherein the spacer group comprises a chain of about 2 to about 18 atoms in length wherein the chain comprises carbon or comprises carbon and at least one heteroatom, (c) the number of molecules of biotin attached to the antibody wherein the number of molecules of biotin in the conjugate is controlled by controlling the molar challenge ratio of a biotin-derivatizing agent to the antibody or the fragment thereof in the preparation of the conjugate and (d) the point of attachment of biotin to the antibody wherein the biotin is attached to amino groups of intact antibody or a fragment thereof or sulfhydryl groups in the hinge region of intact antibody or a fragment. In some embodiments the method comprises preparing two or more conjugates by selecting a set of parameters for each conjugate wherein the set of parameters is different for each conjugate, conducting an assay for the analyte employing each conjugate and selecting for use in the assay the conjugate that provides the optimum assay sensitivity.

### BRIEF DESCRIPTION OF THE DRAWINGS

FIG. 1A is a graph depicting signal (kcounts) versus concentration (U/mL) obtained in assays with biotin reagents prepared from IgG, F(ab′) and F(ab′)₂ reacted with biotin derivatives containing different spacer arms and utilizing different spacer chemistry (biotinylated antibody reagents). FIG. 1A shows performance of these biotinylated reagents in order of decreasing signal at the upper end of the signal range.

FIG. 1B is a graph depicting signal (kcounts) versus concentration (U/mL) obtained in assays with biotin reagents prepared from IgG and F(ab′)₂ reacted with biotin derivatives containing different spacer arms and utilizing different spacer chemistry (biotinylated antibody reagents). FIG. 1B shows performance of these biotinylated reagents in order of decreasing signal at the lower end of the signal range.

### DETAILED DESCRIPTION OF SPECIFIC EMBODIMENTS

### General Discussion

The present methods provide for modulating the sensitivity of analyte signal conjugates for use in assays for the detection of analytes. The methods disclosed herein comprise assay reagents and formats that achieve not only sufficient signal generation but also achieve good performance including sensitivity at the low end of the medical decision range. One can monitor performance at the low end of the medical decision range by carrying out experiments with samples that contain known amounts of an analyte. Such samples are often referred to as calibrators. Typically, the calibrators are tested in the same manner as the testing of a sample suspected of containing an analyte, the amount of which in the sample is usually unknown. The calibrators typically contain differing, but known, concentrations of analyte. Preferably, the concentration ranges present in the calibrators span and exceed the normal range of suspected analyte concentrations in unknown samples. Dilutions may be required for samples exceeding the normal concentration range.

Performance of a particular assay format at the low end of the medical decision range can be monitored by monitoring the difference in the amount of signal obtained for calibrators spanning the suspected concentration range of interest of the analyte. A large difference or separation between the signal for calibrators such as, for example, calibrator level 1 (L1) and calibrator L2 or calibrator L2 and calibrator L3, is desired. For example, six calibrators may be employed, arbitrarily named L1-L6. Signal to noise ratio may be evaluated by determining an amount of signal using a calibrator that contains no analyte, arbitrarily designated calibrator L1 (background), and the amount of signal obtained for a calibrator containing a first known amount of analyte above zero, arbitrarily designated calibrator L2. This evaluation may also include determining an amount of signal using calibrator L1 and the amount of signal for a calibrator containing a second known amount of analyte above zero, arbitrarily designated L3. Such an evaluation may also include such determination using calibrators L4, L5, L6 and so forth. The embodiments discussed herein provide for improved performance in an assay for an analyte compared to reagents not in accordance with the present embodiments.

A large difference between the signal for calibrators, e.g., calibrator L1 and calibrator L2, or calibrator L1 and calibrator L6, is desired to increase the sensitivity of the method. For good sensitivity in the medical decision range, the difference in the signal detected between calibrator L1 and calibrator L2 is at least about 50%, at least about 75%, at least 90%, at least about 100%, at least about 125%, at least about 150%, at least 175%, at least about 200%, at least about 225%, 250%, at least about 275%, at least 300%, at least about 325%, at least about 350%, at least 375%, at least about 400%, at least about 425%, and so forth. In some embodiments the signal detected for calibrator L6 is at least about 10 times, at least about 20 times, at least about 30 times, at least about 40 times, at least about 50 times, at least about 60 times, at least about 70 times, at least about 80 times, at least about 90 times, at least about 100 times, greater than the signal detected for calibrator L1. Depending on the assay format, the difference in signal may be an increase in signal or a decrease in signal. Typically, the results of the assays using the calibrators are presented in a graph format wherein the amount of signal is plotted against the concentration of the calibrators. In accordance with embodiments of the present invention the slope of the line between calibrator L1 and calibrator L2 generally is steeper compared with results obtained with assay reagents not in accordance with the present embodiments. Furthermore, the slope of the line from calibrator L1 to calibrator L6 is usually steeper compared with results obtained with assay reagents not in accordance with the present embodiments.

To achieve the desired performance such as, for example, optimum sensitivity in the medical decision range, the present inventors discovered a method of designing receptor reagents for use in assays for analytes. The design of a receptor reagent for a particular assay system in accordance with the present embodiments involves the discovery that the structure of the spacer group between the small molecule and the receptor for the analyte in the conjugate and/or the location of biotin attachment to the receptor for the analyte, e.g., antibody, are important. Furthermore, the number of molecules of the small molecule in the conjugate also impacts the performance of small molecule-receptor conjugates. The design involves controlling the above factors in the preparation of small molecule-receptor conjugates, which allow the modulation of sensitivity in assays in which the above conjugates are employed. Typically, the modulation results in enhancing the sensitivity of an assay. However, the modulation may also include lowering sensitivity where maximum sensitivity is not desired for one or more reasons such as the nature of the measuring or detecting system, the range of signal detection of the detecting system, saturation of the detection system due to extent of signal generation, a large variation in the analyte concentration present in the sample to be analyzed and the like. The present methods find application when it is necessary to lower excessive sensitivity, to gain overall modulation of a reaction system, and so forth.

Optimum assay sensitivity is an assay sensitivity that is desired for a particular assay system and takes into consideration the above factors. The assay system includes the reagents that are involved in the detection of a particular analyte and usually those reagents that are involved in the formation and detection of a complex of the analyte with a receptor for the analyte. Such reagents include receptors such as antibodies, which may be linked to a small molecule (small molecule-receptor conjugates), labeled with a member of a signal producing system, bound to a support, and the like. The design of a small molecule-receptor reagent in accordance with the present embodiments involves the small molecule-receptor reagent and the manner in which it interacts with the analyte and other reagents of the assay system to produce a desired assay sensitivity.

The present methods find particular application to the situation where an antibody may not be suitable for use in a particular assay system for an analyte for one or more reasons. For example, in the case of an assay system that involves the formation of a complex of an analyte in the form of a sandwich between two antibodies, such reasons include lack of desired sensitivity due to nature of the complex (sandwich) formed between the analyte, the specific antibody and a second analyte-specific antibody, the method of immobilization of the second antibody onto a solid support, the system-specific detection system, the utilization of the intact antibody or a fragment thereof, and so forth. The present methods allow an antibody to be employed to achieve optimum assay sensitivity by preparing a conjugate of the antibody and a small molecule such as, e.g., biotin, where the sensitivity of the assay can be modulated by controlling the aforementioned parameters in the preparation of the conjugate. Consequently, one can achieve a desired assay sensitivity and/or assay range (range of suspected concentration of an analyte in a sample) by employing an existing antibody and one can avoid the sometimes laborious task of developing a new antibody to obtain a desired assay sensitivity.

A receptor is member of a specific binding pair (“sbp member”), which is one of two different molecules having an area on the surface or in a cavity, which specifically binds to and is thereby defined as complementary with a particular spatial and polar organization of the other molecule. The members of the specific binding pair are referred to as ligand and receptor (anti-ligand). These will usually be members of an immunological pair such as antigen-antibody, although other specific binding pairs such as IgG-protein A, and the like are not immunological pairs but may be included.

The receptor of the small molecule-receptor conjugate may be an antibody, a nucleotide, an analyte-specific binding protein, and the like. The antibody may be an antibody for the analyte. By the phrase “antibody for the analyte” is meant an antibody that binds specifically to analyte and does not bind to any significant degree to other entities such that the analysis for analyte would be distorted. Usually, at least one antibody for the analyte is employed either as part of the biotin-receptor conjugate or separately. In some embodiments at least two different antibodies for the analyte may be employed.

Antibodies specific for an analyte for use in immunoassays can be monoclonal or polyclonal. Such antibodies can be prepared by techniques that are well known in the art such as immunization of a host and collection of sera (polyclonal) or by preparing continuous hybrid cell lines and collecting the secreted protein (monoclonal) or by cloning and expressing nucleotide sequences or mutagenized versions thereof coding at least for the amino acid sequences required for specific binding of natural antibodies or by generating ascites using in vivo models.

Antibodies may include a complete or intact immunoglobulin or fragments thereof, which immunoglobulins include the various classes and isotypes, such as IgA, IgD, IgE, IgG1, IgG2a, IgG2b and IgG3, IgM, etc. Fragments thereof may include Fab, Fv and F(ab′)₂, Fab′, and the like. When the IgG is digested enzymatically, different fragments are obtained depending on the enzyme used; for instance, if papain is used, three fragments are obtained, the carbohydrate-containing fragment (Fc) and two antigen-binding fragments (Fab) and, if pepsin is used, one F(ab′)₂ fragment is obtained, while the carbohydrate-containing fragment is digested. The foregoing is due to the fact that papain cuts the heavy chains immediately after the hinge region (towards the amino terminal region), while pepsin cuts them before the hinge (towards the carboxy terminal region). When treated with a reagent capable of reducing disulfide bonds, the F(ab′)₂ fragment is broken into two fragments, called Fab′ that have the same immunological properties as the Fab fragments produced by papain digestion. In addition to intact antibody and antibody fragments, aggregates, polymers, and conjugates of immunoglobulins or of their fragments can be used where appropriate so long as binding affinity for a particular molecule is maintained. The hinge region of an antibody is the region at which the arms of the antibody molecule form a Y. This region is called the hinge region because there is some flexibility in the antibody molecule at this point.

Antiserum containing antibodies (polyclonal) is obtained by well-established techniques involving immunization of an animal, such as a rabbit, sheep, horse, chicken, guinea pig, goat, or the like with an appropriate immunogen and obtaining antisera from the blood of the immunized animal after an appropriate waiting period. State-of-the-art reviews are provided by Parker, Radioimmunoassay of Biologically Active Compounds, Prentice-Hall (Englewood Cliffs, N.J., U.S., 1976), Butler, J. Immunol. Meth. 7: 1-24 (1975); Broughton and Strong, Clin. Chem. 22: 726-732 (1976); and Playfair, et al., Br. Med. Bull. 30: 24-31 (1974).

Antibodies can also be obtained by somatic cell hybridization techniques, such antibodies being commonly referred to as monoclonal antibodies. Monoclonal antibodies may be produced according to the standard techniques of Köhler and Milstein, Nature 265:495-497, 1975. Reviews of monoclonal antibody techniques are found in Lymphocyte Hybridomas, ed. Melchers, et al. Springer-Verlag (New York 1978), Nature 266: 495 (1977), Science 208: 692 (1980), and Methods of Enzymology 73 (Part B): 3-46 (1981). In another approach for the preparation of antibodies, the sequence coding for antibody binding sites can be excised from the chromosome DNA and inserted into a cloning vector, which can be expressed in bacteria to produce recombinant proteins having the corresponding antibody binding sites.

A conjugate is a molecule comprised of two or more substructures bound together covalently, generally through a spacer group, to form a single structure. The binding is by means of an attaching group. For example, a receptor attached to a chain of atoms that connects to a small molecule is a small molecule-receptor conjugate. The spacer group is a portion of a structure that connects two or more substructures such as, for example, a small molecule to a receptor. Conjugation is any process wherein two subunits are coupled together by means of one or more covalent bonds to form a conjugate. The conjugation process can be comprised of any number of steps.

The small molecule is a compound of molecular weight less than about 2000, or less than about 1500, and in the range of about 100 to about 1000, or about 300 to about 600. The small molecule is usually a small organic molecule such as, for example, biotin, dyes such as, e.g., fluorescein, rhodamine and the like, drugs such as, e.g., digoxin, digoxigenin, tetracycline, and the like, vitamins such as, e.g., folate, B12 and the like, and so forth. The term “biotin” includes all entities that have an affinity towards avidin, streptavidin, anti-biotin antibodies or genetically modified proteins that have binding properties similar to that of avidin, streptavidin, anti-biotin antibodies, and the like. The term, therefore, includes biotin, biocytin, desthibiotin, and so forth. The remaining discussion is directed, by way of illustration and not limitation, to biotin as representative of a small molecule.

In the present biotin-receptor conjugates, the biotin is attached to any of multiple amino groups present throughout the protein structure or to sulflhydryl groups in the hinge region of the receptor such as, for example, an antibody, by means of a spacer group that comprises a chain of atoms that is about 2 to about 18 atoms in length, or about 3 to about 16 atoms in length, or about 4 to about 14 atoms in length. The number of atoms of the spacer group may be 2, or 3, or 4, or 5, or 6, or 7, or 8, or 9, or 10, or 11, or 12, or 13, or 14, or 15, or 16, or 17, or 18. In some embodiments the number of atoms in the chain of atoms of the spacer group is 5, or 6, or 7, or 8 (or an integer of 5 to 8). The atoms in the chain of the spacer group may be all carbon or they may comprise one or more heteroatoms selected from the group consisting of oxygen, nitrogen, sulfur, phosphorus, selenium, tungsten, silicon, and so forth. The spacer group may also comprise one or more heteroatoms as substituents on the chain; such heteroatoms include the aforementioned heteroatoms and in addition halogen (chlorine, bromine, iodine), and the like

In some embodiments the carboxylic acid functionality on the biotin can be used for the attachment to the spacer group. The carboxylic acid can be reduced to an aldehyde or alcohol or directly activated for reaction with a corresponding reactive moiety of a derivatizing agent that comprises the spacer group. The derivatizing agent is a reagent that comprises the spacer group where the derivatizing agent is functionalized for reaction with the biotin and/or the antibody. Many methods for activation of the carboxylic acid group are known such as, for example, conversion to an active ester such as a N-hydroxysuccinimide ester, p-nitrophenyl ester, phenyl thioester, and the like; a mixed anhydride such as, for example, by reaction with a chlorocarbonate mixed anhydride; a carboxylic acid halide; activation by a carbodiimide; and so forth.

In some embodiments the biotin having an activated carboxylic acid group may be reacted preferably with a spacer group or a portion thereof that is at least a mono-functionalized or a bi-functionalized derivatizing reagent for connecting to the biotin on the one hand and, in the case of a bi-functionalized reagent, to the receptor or another portion of the spacer group on the other hand. Thus, the activated carboxylic acid group of the biotin can react with available nucleophilic groups such as amines, active methylene groups, alcohols, enamines, etc., on a mono-functionalized or a bi-functionalized reagent. Alternatively, the biotin carboxylic acid can be reduced to an aldehyde and reacted with amines by reductive amination, or with hydrazines, hydroxylamines, hydrazides and the like present in the bi-functionalized reagent. The alcohol produced by reduction of the carboxylic acid of biotin can likewise be reacted with a mono-functionalized reagent or a bi-functionalized reagent by reaction with active esters, alkylating groups such as alpha-haloamides such as, for example, alpha-bromoamides, and the like or the alcohol can be converted to a leaving group such as tosylate or bromide for reaction with groups on a mono-functionalized reagent or a bi-functionalized reagent such as alcohols, amines, hydrazines, thiols, and the like.

A reagent is bi-functionalized in that it has two functionalities for spacer to two components, one being the biotin and the other being the receptor or another portion of the spacer group. The bi-functionalized reagent is comprised of a chain of atoms terminated at each end in a functional group designed to react with the activated biotin on the one hand and the receptor or another portion of the spacer group on the other hand. When the spacer group is prepared from one or more smaller components, the bi-functionalized reagent is prepared from smaller molecules containing a lower number of atoms wherein the molecules become covalently bound by virtue of various functionalities mentioned above. For example, carboxylic acid groups, and their nitrogen, e.g., imidate, and sulfur, e.g., isothiocyanate, analogs may be linked to available amino groups as discussed immediately above. The carbonyl of a keto group can be condensed directly with an amino group. An alcohol functionality can react with an anhydride to form a mono ester. The free carboxy group can then be activated by preparing the mixed anhydride and be used for reaction with an amino group. An alpha-haloacetamide, for example, can be formed from an amino group and used to form a carbon-nitrogen bond by reaction with a molecule containing a free amino group. In other embodiments the alpha-haloacetamide, for example, can be formed from an amino group and used to form a carbon-sulfur bond by reaction with a sulfhydryl-containing molecule. The alpha-haloacetamide may be, for example, alpha-bromoacetamide, alpha-iodoacetamide, and the like. In other embodiments, an alpha-haloacetyl functionality may be employed such as, for example, alpha-bromoacetyl, alpha-iodoacetyl, and so forth.

In some embodiments a spacer group is attached first to the biotin such as in the instance of a mono-functionalized reagent. The spacer group is then functionalized for attachment to an antibody. For example, the spacer group may comprise a functionality from which an N-hydroxysuccinimidyl ester may be prepared for reaction with free amino groups of an antibody forming a stable amide linkage. In some embodiments an antibody may be treated with a reducing agent such as, for example, dithiothreitol, dithioerythritol, tris(2-carboxyethyl)phosphine hydrochloride or the like to form free sulfhydryl groups. In these embodiments the spacer group of the biotin reagent may comprise maleimido, epoxy or haloacetyl such as, for example, iodoacetyl, functionalities for reaction with the free sulfhydryl groups of the antibody to form a stable thioether.

As mentioned above, in some embodiments the spacer group comprises only carbon atoms in the chain although the carbon atoms of the chain may comprise one or more atoms other than hydrogen such as heteroatoms in the form of hydroxyl, amino, aldehyde, carboxyl, thiol, ether, thioether, azido, epoxy, silane, halogen and the like. When the spacer group comprises only carbon atoms in the chain of atoms and comprises only hydrogen as the other atoms on the carbon atoms, the spacer group, in some embodiments, is about 2 to about 18 carbon atoms in length, or about 3 to about 16 carbon atoms in length, or about 4 to about 12 carbon atoms in length, or about 5 to about 10 carbon atoms in length, or about 6 to about 9 carbon atoms in length. The atoms in the spacer group may be present in saturated (alkyl-derived) or unsaturated (alkenyl-derived and alkynyl-derived) form or combinations thereof. The spacer group may comprise atoms present in the form of double bonds, triple bonds, cycloalkyl (C₃ to C₇), phenyl, and so forth. In some embodiments, such a spacer group may be represented by the formula:

—CH₂(CH₂)ₙCH₂— (I) wherein n is 0 to 16, or 1 to 14, or 2 to 10 or 3 to 8, or 4 to 7; in some embodiments n is 4; or
—CH₂(CH═CH)ₘCH₂—(CH₂)_{q}—CH₂—(CH═CH)ₘCH₂— (II) wherein m is independently 0 to 2, or 1 to 2, or 0 to 1 with the proviso that at least one of m is not 0, and q is 0 to 6, or 1 to 6, or 2 to 6, or 2 to 5, or 2 to 4, or 3 to 6, or 3 to 5, with the proviso that the total number of carbon atoms in the chain not be greater than 18; in some embodiments m is 1 and 0, respectively, and q is 0; or
—CH₂(CH≡CH)ₛCH₂—(CH₂)ᵣ—CH₂(CH≡CH)ₛCH₂— (III) wherein s is independently 0 to 2, or 1 to 2, or 0 to 1 with the proviso that at least one of s is not 0, and r is 0 to 6, or 1 to 6, or 2 to 6, or 2 to 5, or 2 to 4, or 3 to 6, or 3 to 5, with the proviso that the total number of carbon atoms in the chain not be greater than 18; in some embodiments s is 1 and 0, respectively, and r is 0; or

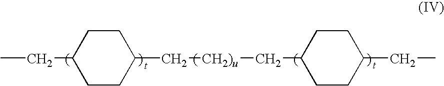

wherein t is independently 0 to 2, or 1 to 2, or 0 to 1 with the proviso that at least one of t is not 0, and u is 0 to 6, or 1 to 6, or 2 to 6, or 2 to 5, or 2 to 4, or 3 to 6, or 3 to 5, with the proviso that the total number of carbon atoms in the chain not be greater than 18; in some embodiments t is 1 and 0, respectively, and u is 0; or

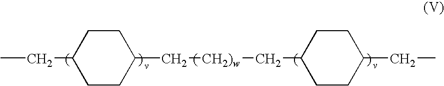

wherein v is independently 0 to 2, or 1 to 2, or 0 to 1 with the proviso that at least one of v is not 0, and w is 0 to 6, or 1 to 6, or 2 to 6, or 2 to 5, or 2 to 4, or 3 to 6, or 3 to 5, with the proviso that the total number of carbon atoms in the chain not be greater than 18; in some embodiments v is 1 and 0, respectively, and w is 0.

For purposes of this disclosure, “alkyl-derived” means a linear saturated divalent hydrocarbon radical or a branched saturated divalent hydrocarbon radical having the number of carbon atoms indicated in the prefix. By way of example, (C₂)-alkyl-derived means —CH₂CH₂—, (C₃)-alkyl-derived means —CH₂CH₂CH₂—, and branched isomers thereof, and so forth. “Alkenyl-derived” means a linear monovalent hydrocarbon radical or a branched monovalent hydrocarbon radical having the number of carbon atoms indicated in the prefix and containing at least one double bond. By way of example, (C₂)-alkenyl-derived means —CH═CH—, (C₃)— alkenyl-derived means —CH═CHCH₂—, (C₄)— alkenyl-derived means —CH═CHCH₂CH₂— or —CH₂CH═CHCH₂—, and branched isomers thereof, and so forth. “Alkynyl-derived” means a linear monovalent hydrocarbon radical or a branched monovalent hydrocarbon radical containing at least one triple bond and having the number of carbon atoms indicated in the prefix. For example, (C₂)-alkynyl-derived means —C≡C—, (C₃)— alkynyl-derived means —C≡CCH₂—, (C₄)— alkynyl-derived means —C≡CCH₂CH₂— or —CH₂C≡CCH₂—, and branched isomers thereof, and so forth.

As mentioned above, in some embodiments the spacer group comprises a chain of atoms, which are carbon atoms and one or more heteroatoms. The heteroatoms present in the chain may be in the form of an ether, ester, secondary or tertiary amine, amide, thioether, thioesters, selenide, silane and so forth. In some embodiments the carbon atoms of the chain may comprise one or more atoms other than hydrogen such as heteroatoms in the form of hydroxyl, amino, aldehyde, carboxyl, thiol, ether, thioether, azido, epoxy, silane, and the like. When the spacer group comprises only carbon atoms and oxygen atoms in the chain of atoms and comprises only hydrogen as the other atoms on the carbon atoms, the spacer group, in some embodiments, is an alkyl-derived ether, an alkenyl-derived ether or an alkynyl-derived ether comprising 2 to about 6, or about 2 to about 5, or about 2 to about 4, oxygen atoms. The length of such a spacer group is about 2 to about 18 atoms in length, or about 3 to about 16 atoms in length, or about 4 to about 12 atoms in length, or about 5 to about 10 atoms in length, or about 6 to about 9 atoms in length. In some embodiments the spacer group comprises one or more ethylene oxide units and is represented by the formula:

—CH₂(CH₂CH₂O)ₚ— wherein p is 1 to 5, or 1 to 4, or 1 to 3, or 1 to 2, or 2 to 5, or 2 to 4, or 2 to 3, or 3 to 5, or 3 to 4, or 4 to 5. In some embodiments, p is 2 and in some embodiments p is 4.

Compounds of the present embodiments can be prepared by a combination of general procedures that are known in the art. Activated biotin reagents are described in the literature and some are commercially available. Spacer groups can be formed in situ or may be prepared prior to attachment of the spacer to the biotin and to the sbp member. In some embodiments, biotin having the desired spacer group is prepared and this entity is reacted with the sbp member.

In embodiments where the antibody is a Fab′ fragment, the biotin may be linked to the antibody by means of a spacer group that comprises carbon and at least one heteroatom selected from the group consisting of oxygen, nitrogen, halogen, silicon and sulfur. Such spacer groups are discussed above. In some embodiments where the antibody is a Fab′ fragment, the biotin may be linked to the antibody by means of one or more amino groups of the antibody and not by means of sulfhydryl groups.

In some embodiments, when the antibody is intact antibody and the biotin is linked to sulfhydryl groups of the antibody, the biotin is linked to the antibody by means of a spacer group that comprises carbon and at least one heteroatom selected from the group consisting of oxygen, nitrogen, halogen, silicon and sulfur. Such spacer groups are discussed above.

In some embodiments the molar challenge ratio of the biotin derivative to the antibody or the fragment thereof is controlled during the synthesis of the biotin-antibody conjugate so that the resultant conjugate, when employed in an assay for an analyte, allows for modulation of the sensitivity of the assay. In some instances, it has been found that incorporation of a limited number of biotin molecules is desirable over the incorporation of an excess number of biotin molecules. The number of biotin molecules incorporated into an antibody molecule may be controlled by the molar challenge ratio, which is the ratio of the number of moles of biotin per moles of antibody employed in the reaction of the biotin moiety with the antibody. The molar challenge ratio employed is dependent on the nature of the antibody such as, for example, intact antibody or antibody fragment, on the nature of the analyte, on the nature of the specific assay system and the format employed to analyze a particular analyte utilizing the antibody-biotin conjugate, the reaction conditions employed during synthesis of the biotin-antibody reagent, and so forth. A particular molar challenge ratio results in a number of biotin molecules incorporated into the conjugate, which is usually determined by carrying out the reaction at a particular molar challenge ratio and determining the number of biotin molecules incorporated. Typically, the number of biotin molecules incorporated is dependent on the pH of the reaction mixture used during the synthesis of the antibody-biotin reagent. For example, the number of biotins incorporated at pH 7.0 is usually less than the molar challenge ratio by a factor of about 3 to about 4.

In some embodiments the molar challenge ratio (the ratio of biotinylation reagent:antibody) is about 1:1 to about 30:1, or about 1:1 to about 25:1, or about 1:1 to about 20:1, or about 1:1 to about 15:1, or about 1:1 to about 10:1, or about 1:1 to about 9:1, or about 1:1 to about 8:1, or about 1:1 to about 7:1, or about 1:1 to about 6:1, or about 1:1 to about 5:1, or about 1:1 to about 4:1, or about 1:1 to about 3:1 or about 1:1 to about 2:1. In some embodiments an equimolar amount of biotin reagent is reacted with the antibody. In some embodiments the number of biotin molecules incorporated into the biotin-antibody conjugate is about 1:1 to about 5:1, or about 1:1 to about 4:1, or about 1:1 to about 3:1, or about 1:1 to about 2:1, or about 1:1 to about 1.5:1, or about 1.5:1 to about 5:1, or about 1.5:1 to about 4:1, or about 1.5:1 to about 3:1, or about 1.5:1 to about 2:1, or about 2:1 to about 5:1, or about 2:1 to about 4:1, or about 2:1 to about 3:1 or about 0.5:1 to about 5:1, or about 0.5:1 to about 4:1, or about 0.5:1 to about 3:1, or about 0.5:1 to about 2:1, or about 0.5:1 to about 1.5:1, or about 0.5:1 to about 1:1.

Control of the number of biotins incorporated into the biotin-antibody conjugate is also dependent on the pH of the reaction between the biotin that is derivatized with the functionalized agent comprising the spacer group for reaction with the antibody. The pH for this reaction is dependent on the nature of the functionalized reagent, whether the functionalized reagent is reacted with amino groups or sulfhydryl groups, and so forth. The pH may be about 6.0 to about 8.0, or about 6.5 to about 7.5. In some embodiments, the pH is less than 8.0, or less than 7.9, or less than 7.8, or less than 7.7 or less than 7.6, or less than 7.5, or less than 7.4, or less than 7.3, or less than 7.2, or less than 7.1 and greater than 6.9; usually in the range of about 7.0 to about 7.9, or about 7.0 to about 7.8, or about 7.0 to about 7.7, or about 7.0 to about 7.6, or about 7.0 to about 7.5, or about 7.0 to about 7.4, or about 7.0 to about 7.3, or about 7.0 to about 7.2, or about 7.0 to about 7.1.

In some embodiments the sensitivity of an assay system may be modulated by controlling the hydrophilic or hydrophobic nature of a spacer group that links biotin to the antibody to form the biotin-antibody conjugate reagent employed as an assay reagent in the assay system. In these embodiments the hydrophobicity or hydrophilicity of the spacer group is adjusted based on the performance of the biotin-antibody conjugate in an assay for an analyte. The particular spacer group is chosen by conducting assays utilizing biotin-antibody conjugates with spacer groups of differing hydrophobicity and/or hydrophilicity and selecting the biotin-antibody conjugate that yields the desired performance such as assay range and achieved sensitivity in the assay.

The term “hydrophobic” refers to a molecule that is non-polar and thus prefers neutral molecules or non-polar molecules and prefers non-polar solvents. Hydrophobic molecules have an affinity for other hydrophobic moieties compared to hydrophilic moieties. Hydrophobic spacer groups generally are composed of primarily carbon and hydrogen such as, for example, a spacer group comprising primarily alkyl-derived, alkenyl-derived or alkynyl-derived moieties, in either open chain or cyclic form, of about 2 to about 18 carbon atoms in length. Specific embodiments of hydrophobic spacer groups include, for example, compounds of formulas I, II, III, IV and V set forth above.

The term “hydrophilic” refers to a molecule that is polar and usually capable of hydrogen bonding enabling it to dissolve more readily in polar solvents such as water than in non-polar solvents such as oil or other hydrophobic solvents. Hydrophilic molecules have an affinity for other hydrophilic moieties compared to hydrophobic moieties. Hydrophilic spacer groups generally are composed of carbon and hydrogen and one or more heteroatoms such as listed above so that the resulting spacer group has polar characteristics. The polar moieties formed by the heteroatoms include ethers, esters, amines, amides, thioethers, thioesters, alcohols, carboxylic acids, sulfonic acids and phosphoric acids and the like. Specific embodiments of hydrophilic spacer groups include, for example, polyethylene oxide polymers such as —CH₂(CH₂CH₂O)ₘ— wherein m is 1 to 5, or 1 to 4, or 1 to 3, or 1 to 2, or 2 to 5, or 2 to 4, or 2 to 3, or 3 to 5, or 3 to 4, or 4 to 5.

The term “hydrophobic analyte” as used herein refers to an analyte that exhibits a characteristic of interaction by a lipophilic moiety such as, for example, a lipoprotein, or of reduced solubility in a polar medium such as water. Examples of hydrophobic analytes include, by way of illustration and not limitation, immunosuppressant drugs, cancer antigens, steroid hormones (e.g. testosterone and progesterone), drugs of abuse (e.g. benzodiazepine and buprenorphine), thyroid hormones (e.g. thyroxine and tri-iodothyronine) and so forth. Immunosuppressive drugs can be classified as follows: glucocorticoids, cytostatics, drugs acting on immunophilins, and other drugs such as interferons, opiates INF binding proteins, mycophenolate, FTY720 and the like. A particular class of immunosuppressant drugs comprises those drugs that act on immunophilins. Two distinct families of immunophilins are currently known: cyclophilins and macrophilins, the latter of which specifically bind, for example, tacrolimus or sirolimus. The immunosuppressant drugs that act on immunophilin include, for example, cyclosporin (including cyclosporin A, cyclosporin B, cyclosporin C, cyclosporin D, cyclosporin E, cyclosporin F, cyclosporin G, cyclosporin H, cyclosporin I), tacrolimus (FK506, PROGRAF®), sirolimus (rapamycin, RAPAMUNE®), everolimus (RAD, CERTICAN®) and so forth.

The term “hydrophilic analyte” as used herein refers to an analyte that exhibits a characteristic of adsorption by a hydrophilic moiety or of enhanced solubility in a polar medium such as water. Specific examples of hydrophilic analytes include, for example, vancomycin, vitamin B12, hemoglobin, ferritin, insulin, proteins (e.g. hormones, enzymes) and so forth.

The present methods provide for modulation of the sensitivity of an assay for an analyte by controlling certain parameters in the preparation of an antibody reagent that is employed in the assay. As discussed above, the antibody reagent is a conjugate of a small molecule, e.g. biotin, and a receptor, e.g., antibody, for the analyte. Modulation of assay sensitivity may be realized by preparing two or more conjugates wherein a set of parameters enumerated above is selected for each conjugate. The set of parameters is chosen from the aforementioned list of parameters and the set is different for each conjugate. Assays for the analyte are conducted employing each conjugate and the results are analyzed. Based on the results, a conjugate that provides optimum assay sensitivity, i.e., a desired assay sensitivity for the assay system in question, is selected as a reagent for use in future assays for the analyte. The unique combination of both loading ratio and chemical properties of the biotinylating reagent employed (i.e. hydrophobic or hydrophilic) is used to modulate the assay. The assay employed for the selection of an antibody reagent that provides optimum assay sensitivity may be any of the assay methods discussed in more detail below.

### General Description of Assays for an Analyte Utilizing the Present Reagents

The biotin-receptor conjugate reagents described above can be used in specific binding assays for analytes. In some embodiments such assays involve a biotin-binding partner, which may be irreversibly attached to a support or a label or an sbp member. In such an approach the biotin-binding partner is bound to the support or the label or the sbp member and the present reagent then binds to the biotin-binding partner prior to, during or after use of the present biotin-receptor conjugates in an assay. The “biotin-binding partner” may be any moiety that binds to biotin such as, for example, avidin, streptavidin, genetically modified proteins with similar binding properties as that of avidin and anti-biotin antibody and the like.

The reagents of the present embodiments may be used in most assays for the determination of an analyte that is an sbp member. In general, in such assays the reagents comprise, among others, a receptor for the analyte. A sample suspected of containing an analyte is combined in an assay medium with a receptor for the analyte. The binding of the receptor to the analyte, if present, is detected. The receptor for the analyte may be the receptor of the present conjugates. The assay can be performed either without separation (homogeneous) or with separation (heterogeneous) of any of the assay components or products.

The present reagents may be used in many types of immunoassays to determine the presence and/or amount of an analyte in a sample suspected of containing such analytes. The immunoassays may involve labeled or non-labeled reagents. Immunoassays involving non-labeled reagents usually comprise the formation of relatively large complexes involving one or more antibodies. Such assays include, for example, immunoprecipitation and agglutination methods and corresponding light scattering techniques such as, e.g., nephelometry and turbidimetry, for the detection of antibody complexes. Labeled immunoassays include enzyme immunoassays, fluorescence polarization immunoassays, radioimmunoassay, inhibition assay, induced luminescence, fluorescent oxygen channeling assay, and so forth.

One general group of immunoassays that may be employed includes immunoassays using a limited concentration of antibody. Another group of immunoassays involves the use of an excess of one or more of the principal reagents such as, for example, an excess of an antibody for the analyte. Another group of immunoassays are separation-free homogeneous assays in which the labeled reagents modulate the label signal upon analyte-antibody binding reactions. Another group of assays includes labeled antibody reagent limited competitive assays for analyte that avoid the use of haptens that pose a chemical challenge for labeling. In this type of assay, the solid phase immobilized analyte is present in a constant, limited amount. The partition of a label between the immobilized analyte and free analyte depends on the concentration of analyte in the sample.

The assays can be performed either without separation (homogeneous) or with separation (heterogeneous) of any of the assay components or products. Homogeneous immunoassays are exemplified by the EMIT® assay (Siemens Healthcare Diagnostics Inc., Deerfield, Ill.) disclosed in Rubenstein, et al., U.S. Pat. No. 3,817,837, column 3, line 6 to column 6, line 64; immunofluorescence methods such as those disclosed in Ullman, et al., U.S. Pat. No. 3,996,345, column 17, line 59, to column 23, line 25; enzyme channeling immunoassays (“ECIA”) such as those disclosed in Maggio, et al., U.S. Pat. No. 4,233,402, column 6, line 25 to column 9, line 63; the fluorescence polarization immunoassay (“FPIA”) as disclosed, for example, in, among others, U.S. Pat. No. 5,354,693; enzyme immunoassays such as the enzyme linked immunosorbant assay (“ELISA”). Exemplary of heterogeneous assays are the radioimmunoassay, disclosed in Yalow, et al., J. Clin. Invest. 39:1157 (1960). The above disclosures are all incorporated herein by reference.

Other enzyme immunoassays are the enzyme modulate mediated immunoassay (“EMMIA”) discussed by Ngo and Lenhoff, FEBS Lett. (1980) 116:285-288; the substrate labeled fluorescence immunoassay (“SLFIA”) disclosed by Oellerich, J. Clin. Chem. Clin. Biochem. (1984) 22:895-904; the combined enzyme donor immunoassays (“CEDIA”) disclosed by Khanna, et al., Clin. Chem. Acta (1989) 185:231-240; homogeneous particle labeled immunoassays such as particle enhanced turbidimetric inhibition immunoassays (“PETINIA”), particle enhanced turbidimetric immunoassay (“PETIA”), etc.; and the like.

Other assays include the sol particle immunoassay (“SPIA”), the disperse dye immunoassay (“DIA”); the metalloimmunoassay (“MIA”); the enzyme membrane immunoassays (“EMIA”); luminoimmunoassays (“LIA”); and so forth. Other types of assays include immunosensor assays involving the monitoring of the changes in the optical, acoustic and electrical properties of an antibody-immobilized surface upon the binding of a hydrophobic drug. Such assays include, for example, optical immunosensor assays, acoustic immunosensor assays, semiconductor immunosensor assays, electrochemical transducer immunosensor assays, potentiometric immunosensor assays, amperometric electrode assays, and the like.

Heterogeneous assays usually involve one or more separation steps and can be competitive or non-competitive. A variety of competitive and non-competitive heterogeneous assay formats are disclosed in Davalian, et al., U.S. Pat. No. 5,089,390, column 14, line 25 to column 15, line 9, incorporated herein by reference. In a typical competitive heterogeneous assay, a support having an antibody for analyte bound thereto is contacted with a medium containing the sample and analyte analog conjugated to a detectable label such as an enzyme. Analyte in the sample competes with the analyte analog for binding to the antibody. After separating the support and the medium, the label activity of the support or the medium is determined by conventional techniques and is related to the amount of analyte in the sample.

An “analyte analog” is a modified drug that can compete with the analogous analyte for a receptor, the modification providing means to join an analyte analog to another molecule. The analyte analog will usually differ from the analyte by more than replacement of a hydrogen with a bond which links the drug analog to a hub or label, but need not. The analyte analog binds to the receptor in a manner similar to the binding of analyte to the receptor. The analyte analog may be, for example, the analyte conjugated to another molecule through a spacer group, an antibody directed against the idiotype of an antibody to the analyte, and so forth.

A typical non-competitive sandwich assay is an assay disclosed in David, et al., U.S. Pat. No. 4,486,530, column 8, line 6 to column 15, line 63, incorporated herein by reference. In this method, an immune sandwich complex is formed in an assay medium. The complex comprises the analyte, a first antibody (monoclonal or polyclonal) that binds to the analyte and a second antibody that binds to the analyte or a complex of the analyte and the first antibody. Subsequently, the immune sandwich complex is detected and is related to the amount of analyte in the sample. The immune sandwich complex is detected by virtue of the presence in the complex of a label wherein either or both the first antibody and the second antibody contain labels or substituents capable of combining with labels.

Sandwich assays find use for the most part in the detection of analytes, which may be antigens or receptors. In the assay the analyte is bound by two antibodies specific for the analyte and, thus, the assay is also referred to as the two-site immunometric assay. In one approach a first incubation of unlabeled antibody coupled to a support, otherwise known as the insolubilized antibody, is contacted with a medium containing a sample suspected of containing the analyte. After a wash and separation step, the support is contacted with a medium containing the second antibody, which generally contains a label, for a second incubation period. The support is again washed and separated from the medium and either the medium or the support is examined for the presence of label. The presence and amount of label is related to the presence or amount of the analyte. For a more detailed discussion of this approach, see U.S. Pat. Nos. Re 29,169 and 4,474,878, the relevant disclosures of which are incorporated herein by reference.

In a variation of the above sandwich assay the sample in a suitable medium is contacted with labeled antibody for the analyte and incubated for a period of time. Then, the medium is contacted with a support to which is bound a second antibody for the analyte. After an incubation period, the support is separated from the medium and washed to remove unbound reagents. The support or the medium is examined for the presence of the label, which is related to the presence or amount of analyte. For a more detailed discussion of this approach, see U.S. Pat. No. 4,098,876, the relevant disclosure of which is incorporated herein by reference.

In another variation of the above, the sample, the first antibody bound to a support and the labeled antibody are combined in a medium and incubated in a single incubation step. Separation, wash steps and examination for label are as described above. For a more detailed discussion of this approach, see U.S. Pat. No. 4,244,940, the relevant disclosure of which is incorporated herein by reference.

The above assays may be adapted to employ the present reagents. For example, the insolubilized antibody can be formed by combining a biotin-binding partner bound to a support with the biotin-antibody conjugate in accordance with the invention. This may be done prior to, during or after the immune complexation reactions. Alternatively, or in conjunction therewith, a labeled antibody can also be formed by combining avidin bound to a label with a biotin-antibody conjugate as described above. In another approach the second antibody can be unlabeled and a third antibody for the second antibody can be used. In this approach the third antibody may be the antibody of the biotin-antibody conjugate of the present embodiments and a biotin-binding partner is bound to a label.

The present biotin-receptor conjugate reagents can be utilized in any of the known situations wherein a biotin reagent is employed. For example, U.S. Pat. No. 4,298,685 (the relevant disclosure of which is incorporated herein by reference) discloses an assay for an analyte that is an antigen, hapten or other biological substance. A sample suspected of containing the analyte is mixed with antibody for the analyte, which is bound to biotin, and with a known amount of the analyte labeled with an enzyme. After the competitive complexation of the antibody with the labeled analyte and the analyte in the sample, avidin immobilized on an inert support is added. The avidin binds to the biotin and causes the complex to be immobilized on the inert support. After separation of the solid and liquid phases, enzyme activity of one or both is measured, the amount thereof being related to the amount of analyte in the sample. In accordance with the present invention, a biotin-antibody conjugate of the present embodiments can be substituted for the above biotin reagent.

Another example is found in U.S. Pat. No. 4,535,057 (the relevant disclosure of which is incorporated herein by reference), which discloses an immunoassay for determining a viral antigen such as herpes simplex. The antigen is immunocaptured by an insoluble matrix to which is bound antibody for the antigen. Then, the matrix is contacted with a biotin reagent wherein biotin is conjugated to a second antibody for the antigen followed by contact with an avidin reagent wherein avidin is conjugated to a detectable label. If the antigen is present, it binds to the antibody on the matrix. The subsequently added biotin reagent binds to the antigen captured on the matrix and the avidin reagent binds to the biotin. The label is detected as an indication of the presence or amount of the antigen. In the improvement provided by the present invention the above-described biotin-antibody conjugates can be utilized in place of the biotin reagent of the known assay. In the above approach, the antibody of the biotin-antibody conjugate is the second antibody for the antigen.

The present invention has application in the induced luminescence immunoassay referred to in U.S. Pat. No. 5,340,716 (Ullman, et al.) entitled “Assay Method Utilizing Photoactivated Chemiluminescent Label” (“induced luminescence assay”), which disclosure is incorporated herein by reference. In one approach the assay uses a particle incorporating a photosensitizer and a label particle incorporating a chemiluminescent compound. The label particle is conjugated to an sbp member that is capable of binding to an analyte to form a complex, or to a second sbp member to form a complex, in relation to the presence of the analyte. If the analyte is present, the particles containing photosensitizer and the particles containing chemiluminescent compound come into close proximity. The photosensitizer component generates singlet oxygen and activates the chemiluminescent compound when the two particles are in close proximity. The activated chemiluminescent compound subsequently produces light. The amount of light produced is related to the amount of the complex formed, which in turn is related to the amount of analyte present.

In some embodiments of the above assay format, a particle is employed, which comprises the chemiluminescent compound associated therewith such as by incorporation therein or attachment thereto. The particles are conjugated to avidin. An sbp member that binds to the analyte is a biotin-receptor conjugate of the present embodiments. Incubation of the above reagents yields a single reagent wherein the sbp member is bound to the particle in an irreversible manner. Biotin may then be added in an amount sufficient to react with any remaining unoccupied avidin binding sites. A second sbp member that binds to the analyte is part of a biotin-receptor conjugate in accordance with the present embodiments. Avidin is conjugated to a second set of particles having a photosensitizer associated therewith. Incubation of these reagents results in a single reagent having the second sbp member bound to the photosensitizer particles in an irreversible manner. Again, biotin may be added to react with unoccupied avidin binding sites. The reaction medium is incubated to allow the particles to bind to the analyte by virtue of the binding of the sbp members to the analyte. Then, the medium is illuminated with light to excite the photosensitizer, which is capable in its excited state of activating oxygen to a singlet state. Because the chemiluminescent compound of one of the sets of particles is now in close proximity to the photosensitizer by virtue of the presence of the analyte, it is activated by the singlet oxygen and emits luminescence. The medium is then examined for the presence and/or the amount of luminescence or light emitted, the presence thereof being related to the presence of the analyte.

The present invention also finds use in agglutination assays employing plastic particles such as latex particles. In a typical agglutination assay of this type, an sbp member is bound to the surface of the plastic particles. This sbp member is capable of binding to an analyte. Usually, the sbp member is an antigen and the analyte is an antibody. The particles are incubated with a medium suspected of containing the analyte. The presence of the analyte causes the particles to agglutinate and the extent of agglutination is measured by known means and related to the presence or amount of the analyte. The present methods can be used to prepare the particles having the sbp member bound thereto. Avidin can be conjugated to the particles, which can be incubated with a biotin-sbp member conjugate of the present embodiments wherein the sbp member of the conjugate is the sbp member that binds to the analyte. The resulting particles have the sbp member bound thereto in an irreversible manner.

In many of the assays discussed herein, a label is employed; the label is usually part of a signal producing system (“sps”). The nature of the label is dependent on the particular assay format. An sps usually includes one or more components, at least one component being a detectable label, which generates a detectable signal that relates to the amount of bound and/or unbound label, i.e. the amount of label bound or not bound to the analyte being detected or to an agent that reflects the amount of the analyte to be detected. The label is any molecule that produces or can be induced to produce a signal, and may be, for example, a fluorescer, radiolabel, enzyme, chemiluminescer or photosensitizer. Thus, the signal is detected and/or measured by detecting enzyme activity, luminescence, light absorbance or radioactivity, and so forth, as the case may be.

Suitable labels include, by way of illustration and not limitation, enzymes such as alkaline phosphatase, glucose-6-phosphate dehydrogenase (“G6PDH”) and horseradish peroxidase; ribozyme; a substrate for a replicase such as QB replicase; promoters; dyes; fluorescers, such as fluorescein, isothiocyanate, rhodamine compounds, phycoerythrin, phycocyanin, allophycocyanin, o-phthaldehyde, and fluorescamine; complexes such as those prepared from CdSe and ZnS present in semiconductor nanocrystals known as Quantum dots; chemiluminescers such as isoluminol; sensitizers; coenzymes; enzyme substrates; radiolabels such as ¹²⁵l, ¹³¹l, ¹⁴C, ³H, ⁵⁷Co and ⁷⁵Se; particles such as latex particles, carbon particles, metal particles including magnetic particles, e.g., chromium dioxide (CrO₂) particles, and the like; metal sol; crystallite; liposomes; cells, etc., which may be further labeled with a dye, catalyst or other detectable group. Suitable enzymes and coenzymes are disclosed in Litman, et al., U.S. Pat. No. 4,275,149, columns 19-28, and Boguslaski, et al., U.S. Pat. No. 4,318,980, columns 10-14; suitable fluorescers and chemiluminescers are disclosed in Litman, et al., U.S. Pat. No. 4,275,149, at columns 30 and 31; which are incorporated herein by reference.

The label can directly produce a signal and, therefore, additional components are not required to produce a signal. Numerous organic molecules, for example fluorescers, are able to absorb ultraviolet and visible light, where the light absorption transfers energy to these molecules and elevates them to an excited energy state. This absorbed energy is then dissipated by emission of light at a second wavelength. Other labels that directly produce a signal include radioactive isotopes and dyes.

Alternately, the label may need other components to produce a signal, and the signal producing system would then include all the components required to produce a measurable signal. Such other components may include substrates, coenzymes, enhancers, additional enzymes, substances that react with enzymic products, catalysts, activators, cofactors, inhibitors, scavengers, metal ions, and a specific binding substance required for binding of signal generating substances. A detailed discussion of suitable signal producing systems can be found in Ullman, et al., U.S. Pat. No. 5,185,243, columns 11-13, incorporated herein by reference.

Enzymes of particular interest as label proteins are redox enzymes, particularly dehydrogenases such as glucose-6-phosphate dehydrogenase, lactate dehydrogenase, etc., and enzymes that involve the production of hydrogen peroxide and the use of the hydrogen peroxide to oxidize a dye precursor to a dye. Particular combinations include saccharide oxidases, e.g., glucose and galactose oxidase, or heterocyclic oxidases, such as uricase and xanthine oxidase, coupled with an enzyme which employs the hydrogen peroxide to oxidize a dye precursor, that is, a peroxidase such as horse radish peroxidase, lactoperoxidase, or microperoxidase. Additional enzyme combinations are known in the art. When a single enzyme is used as a label, other enzymes may find use such as hydrolases, transferases, and oxidoreductases, preferably hydrolases such as alkaline phosphatase and beta-galactosidase. Alternatively, luciferases may be used such as firefly luciferase and bacterial luciferase.

Illustrative co-factors and co-enzymes that find use include NAD[H], NADP[H], pyridoxal phosphate, FAD[H], FMN[H], etc., usually coenzymes involving cycling reactions. See, for example, U.S. Pat. No. 4,318,980, the disclosure of which is incorporated herein by reference.

In some embodiments the sps has at least first and second sps members. The designation “first” and “second” is completely arbitrary and is not meant to suggest any order or ranking among the sps members or any order of addition of the sps members in the present methods. The sps members may be related in that activation of one member of the sps produces a product, e.g., light, which results in activation of another member of the sps. In some embodiments the sps members comprise a sensitizer and a chemiluminescent composition where activation of the sensitizer results in a product that activates the chemiluminescent composition. The second sps member usually generates a detectable signal that relates to the amount of bound and/or unbound sps member, i.e. the amount of sps member bound or not bound to the analyte being detected or to an agent that reflects the amount of the analyte to be detected.

In some embodiments the first sps member is a sensitizer, such as, for example, a photosensitizer and the second sps member is a chemiluminescent composition that is activated as a result of the activation of the first sps member. The sensitizer may be any moiety that upon activation produces a product that activates the chemiluminescent composition, which in turn generates a detectable signal. In many embodiments the sensitizer is capable of generating singlet oxygen upon activation.

In some embodiments the sensitizer is a photosensitizer for generation of singlet oxygen usually by excitation with light. The photosensitizer can be photoactivatable (e.g., dyes and aromatic compounds) or chemi-activated (e.g., enzymes and metal salts). When excited by light the photosensitizer is usually a compound comprised of covalently bonded atoms, usually with multiple conjugated double or triple bonds. The compound should absorb light in the wavelength range of about 200 to about 1100 nm, or about 300 to about 1000 nm, or about 450 to about 950 nm, with an extinction coefficient at its absorbance maximum greater than about 500 M⁻¹ cm⁻¹, or at least about 5000 M⁻¹ cm⁻¹, or at least about 50,000 M⁻¹ cm⁻¹ at the excitation wavelength. Photosensitizers that are to be excited by light will be relatively photostable and will not react efficiently with singlet oxygen. Several structural features are present in most useful photosensitizers. Most photosensitizers have at least one and frequently three or more conjugated double or triple bonds held in a rigid, frequently aromatic structure. They will frequently contain at least one group that accelerates intersystem crossing such as a carbonyl or imine group or a heavy atom selected from rows 3-6 of the periodic table, especially iodine or bromine, or they may have extended aromatic structures. Typical photosensitizers include acetone, benzophenone, 9-thioxanthone, eosin, 9,10-dibromoanthracene, methylene blue, metallo-porphyrins, such as hematoporphyrin, phthalocyanines, chlorophylis, rose bengal, buckminsterfullerene, etc., and derivatives of these compounds having substituents of 1 to 50 atoms for rendering such compounds more lipophilic or more hydrophilic and/or as attaching groups for attachment, for example, to an sps member or an sbp member.

The photosensitizers useful in the present methods include other substances and compositions that can produce singlet oxygen with or, less preferably, without activation by an external light source. Thus, for example, molybdate salts and chloroperoxidase and myeloperoxidase plus bromide or chloride ion (Kanofsky, J. Biol. Chem. (1983) 259, 5596) have been shown to catalyze the conversion of hydrogen peroxide to singlet oxygen and water. Also included within the scope of the invention as photosensitizers are compounds that are not true sensitizers but which on excitation by heat, light, or chemical activation will release a molecule of singlet oxygen. The best known members of this class of compounds includes the endoperoxides such as 1,4-biscarboxyethyl-1,4-naphthalene endoperoxide, 9,10-diphenylanthracene-9,10-endoperoxide and 5,6,11,12-tetraphenyl naphthalene 5,12-endoperoxide. Heating or direct absorption of light by these compounds releases singlet oxygen. Examples of other photosensitizers that may be utilized are those set forth in U.S. Pat. No. 6,153,442 and U.S. Patent Application Publication No. 20050118727A, the disclosures of which are incorporated herein by reference.

The chemiluminescent composition comprises a substance that undergoes a chemical reaction upon direct or sensitized excitation by light or upon reaction with singlet oxygen or upon chemical activation to form a metastable reaction product that is capable of decomposition with the simultaneous or subsequent emission of light, usually within the wavelength range of about 250 to about 1200 nm. In some embodiments the chemiluminescent composition comprises a substance that reacts with singlet oxygen to form dioxetanes or dioxetanones. The latter are usually electron rich olefins. Exemplary of such electron rich olefins are enol ethers, enamines, 9-alkylidene-N-alkylacridans, arylvinylethers, dioxenes, arylimidazoles, 9-alkylidene-xanthanes and lucigenin. Other compounds include luminol and other phthalhydrazides and chemiluminescent compounds that do not undergo a chemiluminescent reaction by virtue of their being protected by a photochemically labile protecting group, such compounds including, for example, firefly luciferin, aquaphorin, luminol, and the like.

The chemiluminescent compounds preferably emit at a wavelength above 300 nm, preferably above 500 nm, and more preferably above 550 nm. Compounds that absorb and emit light at wavelengths beyond the region where the sample components contribute significantly to light absorption are of particular use in embodiments of the present methods. The electron rich olefins generally have an electron-donating group in conjugation with the olefin. The more preferred olefins are those that yield a dioxetane that decays rapidly at room temperature (less than 60 minutes, preferably less than 5 minutes, desirably less than 30 sec). The dioxetanes may be luminescent alone or in conjunction with a fluorescent energy acceptor. Such olefins include, for example, enol ethers, enamines, 9-alkylidene-N-alkylacridans, 9-alkylidene-xanthanes, 2,3-dihydro-1,4-phthalazinediones, 2,4,5-triphenyl-imidazoles, and the like. Examples of other chemiluminescent compounds that may be utilized are those set forth in U.S. Pat. No. 6,153,442 and U.S. Patent Application Publication No. 20050118727A, the disclosures of which are incorporated herein by reference.

One or more of the biotin-binding moiety, the sbp members and the sps members may be associated with a support. If more than one of the above is associated with a support, the support may be the same or different. For example, where sps members are associated with a support, the same type of support or a different type of support may be employed for each different sps member. As used herein, the phrase “associated with” includes covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, coating one moiety on another moiety, and so forth.

The support may be comprised of an organic or inorganic, solid or fluid, water insoluble material, which may be transparent or partially transparent. The support can have any of a number of shapes, such as particulate including beads and particles, film, membrane, tube, well, strip, rod, planar surfaces such as, e.g., plates, dendrimers, and the like. Depending on the type of assay, the support may or may not be suspendable in the medium in which it is employed. Examples of suspendable supports are polymeric materials such as latex, lipid bilayers or liposomes, oil droplets, cells and hydrogels, magnetic particles, and the like. Other support compositions include polymers, such as nitrocellulose, cellulose acetate, poly(vinyl chloride), polyacrylamide, polyacrylate, polyethylene, polypropylene, poly(4-methylbutene), polystyrene, polymethacrylate, poly(ethylene terephthalate), nylon, poly(vinyl butyrate), etc.; either used by themselves or in conjunction with other materials. Other support compositions include materials such as silica, alumina, glass, silicone and the like.

In some embodiments the supports employed are particles. The particles should have an average diameter of at least about 0.02 microns and not more than about 100 microns. In some embodiments, the particles have an average diameter from about 0.05 microns to about 20 microns, or from about 0.3 microns to about 10 microns. The particle may be organic or inorganic, swellable or non-swellable, porous or non-porous, preferably of a density approximating water, generally from about 0.7 g/mL to about 1.5 g/mL, and composed of material that can be transparent, partially transparent, or opaque. The particles can be biological materials such as cells and microorganisms, e.g., erythrocytes, leukocytes, lymphocytes, hybridomas, streptococcus, Staphylococcus aureus, E. coli, viruses, and the like. The particles can also be particles comprised of organic and inorganic polymers, liposomes, latex particles, magnetic or non-magnetic particles, phospholipid vesicles, chylomicrons, lipoproteins, and the like. In some embodiments, the particles are chrome particles or latex particles.

The polymer particles can be formed from addition or condensation polymers. The particles will be readily dispersible in an aqueous medium and can be adsorptive or functionalizable so as to permit conjugation to an sps member, either directly or indirectly through a spacer group. The particles can also be derived from naturally occurring materials, naturally occurring materials that are synthetically modified, and synthetic materials. Among organic polymers of particular interest are polysaccharides, particularly cross-linked polysaccharides, such as agarose, which is available as Sepharose, dextran, available as Sephadex and Sephacryl, cellulose, starch, and the like; addition polymers, such as polystyrene, polyvinyl alcohol, homopolymers and copolymers of derivatives of acrylate and methacrylate, particularly esters and amides having free hydroxyl functionalities, and the like.

A biotin-binding moiety, an sbp member or an sps member may be associated with a solid support in any manner known in the art. In some embodiments, the sps member may be coated or covalently bound directly to the solid phase or may have layers of one or more carrier molecules such as poly(amino acids) including proteins such as serum albumins or immunoglobulins, or polysaccharides (carbohydrates) such as, for example, dextran or dextran derivatives. Spacer groups may also be used to covalently couple the solid support and the sps member. Other methods of binding the sps members are also possible. The binding of components to the surface of a support may be direct or indirect, covalent or non-covalent and can be accomplished by well-known techniques, commonly available in the literature. See, for example, “Immobilized Enzymes,” Ichiro Chibata, Halsted Press, New York (1978) and Cuatrecasas, J. Biol. Chem., 245:3059 (1970).

The phrase “at least” as used herein means that the number of specified items may be equal to or greater than the number recited. The phrase “about” as used herein means that the number recited may differ by plus or minus 10%; for example, “about 5” means a range of 4.5 to 5.5.

The sample is defined as that which is suspected of containing analyte and which is to be analyzed for the presence or amount of analyte. The samples are preferably from humans or animals and include biological fluids such as whole blood, serum, plasma, sputum, lymphatic fluid, semen, vaginal mucus, feces, urine, spinal fluid, saliva, stool, cerebral spinal fluid, tears, mucus, and the like; biological tissue such as hair, skin, sections or excised tissues from organs or other body parts; and so forth. In many instances, the sample is whole blood, plasma or serum and, in a particular embodiment the sample is serum.

The sample can be prepared in any convenient medium. Conveniently, the sample may be prepared in an assay medium, which is discussed more fully below. In some instances a pretreatment may be applied to the sample such as, for example, to lyse blood cells, and the like. Such pretreatment is usually performed in a medium that does not interfere subsequently with an assay and has no effect on the characteristic properties of the analyte of interest. An aqueous medium is preferred for the pretreatment and typically is one that may be solely water or may include from 0.1 to about 40 volume percent of a cosolvent such as, for example, an organic solvent, which may be an alcohol, ether, ester, and the like. The pH for the pretreatment medium will usually be in the range of about 4 to about 11, more usually in the range of about 5 to about 10, and preferably in the range of about 6.5 to about 9.5.

The assays discussed above are normally carried out in an aqueous buffered medium at a moderate pH, generally that which provides optimum assay sensitivity. The aqueous medium may be solely water or may include from 0.1 to about 40 volume percent of a cosolvent. The pH for the medium will usually be in the range of about 4 to about 11, more usually in the range of about 5 to about 10, and preferably in the range of about 6.5 to about 9.5. The pH will usually be a compromise between optimum binding of the binding members of any specific binding pairs, the pH optimum for other reagents of the assay such as members of the signal producing system, and so forth. Various buffers may be used to achieve the desired pH and maintain the pH during the determination. Illustrative buffers include borate, phosphate, carbonate, tris, barbital, PIPES, HEPES, MES, ACES, MOPS, BICINE, and the like. The particular buffer employed is not critical, but in an individual assay one or another buffer may be preferred.

Various ancillary materials may be employed in the above methods. For example, in addition to buffers the medium may comprise stabilizers for the medium and for the reagents employed. Frequently, in addition to these additives, proteins may be included, such as albumins; organic solvents such as formamide; quaternary ammonium salts; polyanions such as dextran sulfate; binding enhancers, e.g., polyalkyl glycols; or the like. The medium may also comprise agents for preventing the formation of blood clots. Such agents are well known in the art and include, for example, EDTA, EGTA, citrate, heparin, and the like. The medium may also comprise one or more preservatives as are known in the art such as, for example, sodium azide, neomycin sulfate, PROCLIN® 300, Streptomycin, and the like. All of the above materials are present in a concentration or amount sufficient to achieve the desired effect or function. The medium may also comprises one or more detergents such as TRITON®, TWEEN®, ZWITTERGENT®, EP110®, sodium dodecyl sulfate (SDS), BRIJ®, CHAPS®, CHAPSO®, alkylglucosides, NP40® and the like

One or more incubation periods may be applied to the medium at one or more intervals including any intervals between additions of various reagents mentioned above. The medium is usually incubated at a temperature and for a time sufficient for binding of various components of the reagents to occur. Moderate temperatures are normally employed for carrying out the method and usually constant temperature during the period of the measurement. Incubation temperatures normally range from about 5° C. to about 99° C., usually from about 15° C. to about 70° C., more usually 20° C. to about 45° C., preferably about room temperature to about 37° C. The time period for the incubation is about 0.2 seconds to about 24 hours, or about 1 second to about 6 hours, or about 2 seconds to about 1 hour, or about 1 to about 15 minutes. The time period depends on the temperature of the medium and the rate of binding of the various reagents, which is determined by the association rate constant, the concentration, the binding constant and the dissociation rate constant. Temperatures during measurements will generally range from about 10 to about 50° C., or from about 15 to about 40° C.

The concentration of the analyte that may be assayed generally varies from about 10⁻⁵ to about 10⁻¹⁷ M, more usually from about 10⁻⁶ to about 10⁻¹⁴ M. Considerations, such as whether the assay is qualitative, semi-quantitative or quantitative (relative to the amount of the analyte present in the sample), the particular detection technique and the concentration of the analyte normally determine the concentrations of the various reagents.

The concentrations of the various reagents in the assay medium will generally be determined by the concentration range of interest of the analyte, the nature of the assay, and the like. However, the final concentration of each of the reagents is normally determined empirically to optimize the sensitivity of the assay over the range. That is, a variation in concentration of analyte that is of significance should provide an accurately measurable signal difference. Considerations such as the nature of the signal producing system and the nature of the analytes normally determine the concentrations of the various reagents.

As mentioned above, the sample and reagents are provided in combination in the medium. While the order of addition to the medium may be varied, there will be certain preferences for some embodiments of the assay formats described herein. The simplest order of addition, of course, is to add all the materials simultaneously and determine the effect that the assay medium has on the signal as in a homogeneous assay. Alternatively, each of the reagents, or groups of reagents, can be combined sequentially. Optionally, an incubation step may be involved subsequent to each addition as discussed above.

As mentioned above, one specific embodiment of the present invention is a method for determining the presence and/or amount of an analyte in a sample suspected of containing the analyte. A combination is formed in a medium where the combination comprises the sample, a conjugate of biotin and an antibody for the analyte as described above, a biotin-binding moiety wherein the biotin-binding moiety is conjugated to a support, a member of a specific binding pair or a member of a signal producing system, and an analyte analog or a second antibody for the analyte. The combination is subjected to conditions for binding of the analyte to the antibody of the conjugate. The extent of binding of the analyte to the antibody of the conjugate is determined and the extent of the binding is related to the presence and/or amount of the analyte in the sample.

### Examination Step

In a next step of the methods in accordance with the present disclosure, the medium is examined for the presence of a complex comprising the analyte and the antibody for the analyte. The presence and/or amount of the complex indicates the presence and/or amount of the analyte in the sample.

The phrase “measuring the amount of an analyte” refers to the quantitative, semiquantitative and qualitative determination of the analyte. Methods that are quantitative, semiquantitative and qualitative, as well as all other methods for determining the analyte, are considered to be methods of measuring the amount of the analyte. For example, a method, which merely detects the presence or absence of the analyte in a sample suspected of containing the analyte, is considered to be included within the scope of the present invention. The terms “detecting” and “determining,” as well as other common synonyms for measuring, are contemplated within the scope of the present invention.

In many embodiments the examination of the medium involves detection of a signal from the medium. The presence and/or amount of the signal is related to the presence and/or amount of the analyte in the sample. The particular mode of detection depends on the nature of the sps. As discussed above, there are numerous methods by which a label of an sps can produce a signal detectable by external means, desirably by visual examination, and include, for example, electromagnetic radiation, electrochemistry, heat, radioactivity detection, chemical reagents and so forth.

Activation of a signal producing system depends on the nature of the signal producing system members. For an sps member that is a sensitizer that is activated by light, the sps member is irradiated with light. Other activation methods will be suggested to those skilled in the art in view of the disclosures herein.

The examination for presence and/or amount of the signal also includes the detection of the signal, which is generally merely a step in which the signal is read. The signal is normally read using an instrument, the nature of which depends on the nature of the signal. The instrument may be a spectrophotometer, fluorometer, absorption spectrometer, luminometer, chemiluminometer, actinometer, photographic instrument, amperometer, scintillation counter and the like. The presence and amount of signal detected is related to the presence and amount of the analyte present in a sample. Temperatures during measurements generally range from about 10° to about 70° C., or from about 20° to about 45° C., or about 20° to about 25° C. In one approach standard curves are formed using known concentrations of the analytes to be screened. As discussed above, calibrators and other controls may also be used. The dynamic range of an assay relates to the range of signal that the instrument for detection (or detector) is capable of measuring. The present embodiments may be employed to adjust the amount of signal that is obtained so that the amount of signal and/or the change in signal across the assay range of the method falls within the dynamic range of a particular instrument employed in the detection of an analyte.

When a photosensitizer is used, the photosensitizer serves to activate the chemiluminescent composition when the medium containing the above reactants is irradiated. The medium is irradiated with light having a wavelength of sufficient energy to convert the photosensitizer to an excited state and render it capable of activating molecular oxygen to singlet oxygen. When bound to an sbp member, the photosensitizer concentration may be very low, frequently about 10⁻⁶ to about 10⁻¹² M or lower. Generally, for the above embodiments involving a photosensitizer, the medium is irradiated with light having a wavelength of about 300 to about 1200 nm, or about 450 to about 950, or about 550 to about 800 nm.

The period of irradiation will depend on the lifetime of the activated chemiluminescent composition, the light intensity and the desired emission intensity. For short-lived activated chemiluminescent compositions, the period may be less than a second, usually about a millisecond but may be as short as a microsecond where an intense flashlamp or laser is used. For longer-lived activated chemiluminescent compositions, the irradiation period can be longer and a less intense steady light source can be used. In general, the integrated light intensity over the period of irradiation should be sufficient to excite at least 0.1% of the photosensitizer molecules, preferably at least 30%, and, most preferably, every photosensitizer molecule will be excited at least once.

The luminescence or light produced in any of the above approaches can be measured visually, photographically, actinometrically, spectrophotometrically or by any other convenient means to determine the amount thereof, which is related to the amount of analyte in the medium.

A helium-neon laser is an inexpensive light source for excitation at 632.6 nm. Photosensitizers that absorb light at this wavelength are compatible with the emission line of a helium-neon laser and are, therefore, particularly useful in the present methods in which photosensitizers are employed. Other light sources include, for example, other lasers such as Argon, YAG, He/Cd, and ruby; photodiodes; mercury, sodium and xenon vapor lamps; incandescent lamps such as tungsten and tungsten/halogen; and flashlamps.

### Kits Comprising Reagents for Conducting Assays

The present biotin-receptor conjugates and other reagents for conducting a particular assay may be present in a kit useful for conveniently performing an assay for the determination of an analyte. In some embodiments a kit comprises in packaged combination a biotin-antibody for analyte conjugate, streptavidin-sensitizer particles and analyte analog-chemiluminescent particles as well as any other reagents for performing the assay, the nature of which depend upon the particular assay format. In some embodiments a kit comprises antibody for analyte bound to chemiluminescent particles, streptavidin-sensitizer particles and a biotin-antibody for analyte conjugate as well as any other reagents for performing the assay, the nature of which depend upon the particular assay format.

The reagents may each be in separate containers or various reagents can be combined in one or more containers depending on the cross-reactivity and stability of the reagents. The kit can further include other separately packaged reagents for conducting an assay such as additional sbp members, ancillary reagents, and so forth.

The relative amounts of the various reagents in the kits can be varied widely to provide for concentrations of the reagents that substantially optimize the reactions that need to occur during the present method and further to optimize substantially the sensitivity of the assay. Under appropriate circumstances one or more of the reagents in the kit can be provided as a dry powder, usually lyophilized, including excipients, which on dissolution will provide for a reagent solution having the appropriate concentrations for performing a method or assay in accordance with the present invention. The kit can further include a written description of a method in accordance with the present invention as described above.

The following examples further describe the specific embodiments of the invention by way of illustration and not limitation and are intended to describe and not to limit the scope of the invention. Parts and percentages disclosed herein are by volume unless otherwise indicated.

### Other Embodiments

One embodiment is a method of modulating the sensitivity of an assay for the detection of an analyte. The method comprises employing, as a reagent in the assay, a conjugate of a small molecule and an antibody for an analyte wherein the conjugate is prepared by a method wherein the small molecule is attached to amino groups of intact antibody or a fragment thereof or sulfhydryl groups in the hinge region of intact antibody or a fragment thereof by means of a spacer group that comprises a chain of about 2 to about 18 atoms in length wherein the chain comprises carbon or comprises carbon and at least one heteroatom. When the antibody is a Fab′ fragment, the small molecule is linked to the antibody by means of a spacer group wherein the chain comprises carbon and at least one heteroatom or the small molecule is linked to the antibody by means of one or more amino groups of the antibody. In some embodiments the molar challenge ratio of the biotin derivative to the antibody or the fragment thereof is controlled to modulate the sensitivity of the assay. In some embodiments the sensitivity of the assay in question can be modulated by choice of the hydrophilic or hydrophobic nature of the spacer group.

Another embodiment is a method for determining the presence and/or amount of an analyte in a sample suspected of containing the analyte. A combination is formed in a medium where the combination comprises the sample, a conjugate of biotin and an antibody for the analyte, a biotin-binding moiety wherein the biotin-binding moiety is immobilized on a support, a member of a specific binding pair or a member of a signal producing system, and an analyte analog or a second antibody for the analyte. The antibody of the conjugate is intact immunoglobulin, such as intact IgG, or a fragment thereof. In some embodiments, the biotin is linked to amino groups of the antibody or to sulfhydryl groups in the hinge region of the antibody by means of a spacer group that comprises a chain of about 2 to about 18 atoms in length. The chain comprises carbon or comprises carbon and at least one heteroatom. In embodiments where the antibody is a Fab′ fragment, the biotin is linked to the antibody by means of a spacer group wherein the chain comprises carbon and at least one heteroatom or the biotin is linked to the antibody by means of one or more amino groups of the antibody. The combination is subjected to conditions for binding of the analyte to the antibody of the conjugate. The extent of binding of the analyte to the antibody of the conjugate is determined and the extent of the binding is related to the presence and/or amount of the analyte in the sample.

Another embodiment is a method for determining the presence and/or amount of an analyte in a sample suspected of containing the analyte. A combination is provided in a medium wherein the combination comprises the sample, a conjugate of biotin and an antibody for the analyte, a biotin-binding moiety wherein the biotin-binding moiety is conjugated to a support, a member of a specific binding pair or a member of a signal producing system, and an analyte analog or a second antibody for the analyte. In some embodiments the antibody is intact IgG or a fragment thereof and the conjugate is prepared by a method wherein the biotin may be linked to amino groups of the antibody or a fragment thereof by means of a spacer group that comprises a chain of about 2 to about 18 atoms in length wherein the chain comprises carbon or comprises carbon and at least one heteroatom. In some embodiments the antibody is intact IgG and the conjugate is prepared by a method wherein biotin may be linked to sulfhydryl groups in the hinge region of the antibody by means of a spacer group wherein the chain comprises carbon or carbon and at least one heteroatom. In some embodiments the antibody is an antibody fragment and the conjugate is prepared by a method wherein biotin may be linked to sulfhydryl groups in the hinge region of the antibody by means of a spacer group wherein the chain comprises carbon and at least one heteroatom. The combination is subjected to conditions for binding of the analyte to the biotinylated antibody for the analyte. The extent of binding of the analyte to the biotinylated antibody is determined, the extent of the binding being related to the presence and/or amount of the analyte in the sample.

Another embodiment is a reagent for determining the presence and/or amount of an analyte in a sample suspected of containing the analyte. The reagent comprises a conjugate of biotin and an antibody for the analyte. The antibody is intact IgG or a fragment thereof. The biotin is linked to amino groups or sulfhydryl groups in the hinge region of the antibody by means of a spacer group that comprises a chain of about 2 to about 18 atoms in length wherein the chain comprises carbon or comprises carbon and at least one heteroatom. In embodiments where the antibody is a Fab′ fragment, the biotin may be linked to the antibody by means of a spacer group wherein the chain comprises carbon and at least one heteroatom or the biotin is linked to the antibody by means of one or more amino groups of the antibody.

Another embodiment is a reagent for determining the presence and/or amount of an analyte in a sample suspected of containing the analyte, the reagent comprising a conjugate of biotin and an antibody for the analyte wherein the antibody is intact IgG or a fragment thereof and wherein the biotin is linked to amino groups of the antibody or to sulfhydryl groups in the hinge region of the antibody by means of a spacer group that comprises carbon and at least one heteroatom with the proviso that, when the antibody is a Fab′ fragment, the biotin may be linked to the antibody by means of one or more amino groups of the antibody.

### Examples

### Materials

Assays were carried out using the DIMENSION VISTA® analyzer, available from Siemens Healthcare Diagnostics Inc., Deerfield, Ill. The instrument was employed using induced luminescence immunoassay technology and was equipped with an appropriate reader. Unless indicated otherwise reagents were from reagent grade from Sigma/Aldrich, Inc. (Milwaukee, Wis.). Unless indicated otherwise, reactions for the preparation of biotinylated antibody reagents in accordance with the present embodiments were performed at pH 7.0.

Preparation of a F(ab′)₂ fragment. A mixture of 6 mL IgG antibody (5.0 mg/mL in 50 mM HEPES-2 mM EDTA, pH 7.0), 0.11 mL Ficin (10 mg/mL in 50 mM HEPES-2 mM EDTA, pH 7.0; (Sigma Chemical Company, St. Louis Mo.; product #F-4165; EC 3.4.22.3) and 0.073 mL cysteine (10 mg/mL in 50 mM HEPES-2 mM EDTA, pH 7.0; 1 mM final concentration) was heated at 37° C. For this digestion Ficin and antibody are present in 1:30 (w/w) ratio. The reaction mixture was monitored by analytical HPLC (BIOSEP® S3000 column from Phenomenex, Inc., Torrance Calif.) for disappearance of the 150 kDa peak. After 2-3 hr the reaction mixture was quenched by addition of 0.62 mL of NEM (12.5 mg/mL in water; Pierce Chemical Company, product #23030). It was then diluted with an equal volume of 50 mM NaH₂PO₄-300 mM NaCl, pH 7.40 and mixture passed through a PROSEP® High Capacity Protein A column (Millipore Inc., Billerica Mass.) (1×10 cm) using 50 mM NaH₂PO₄-300 mM NaCl, pH 7.40 as elution buffer. Flow through from Protein A column was collected, concentrated and purified by gel filtration on Sephacryl S300 or preparative HPLC using BioSep S3000 column. Fractions containing F(ab′)₂ were collected and concentrated to give 12 mg protein. When the F(ab′)₂ fragment was further reduced to form F(ab′), 100 mM NaH₂PO₄-5 mM EDTA, pH 6.00 (reduction buffer) buffer was used for purification of F(ab′)₂ fragment and when the F(ab′)₂ was used for direct biotinylation, then 10 mM NaH₂PO4-300 mM NaCl, pH 7.00, buffer was used for purification.

Procedure for biotinylation of antibody via amino groups of the antibody. Antibody solution (0.83 mL; 3.0 mg/mL; 16.6 μM) in 10 mM NaH₂PO₄-300 mM NaCl, pH 7.00) was mixed with 29 μL of a 10 mg/mL aqueous solution of NHS-PEO₄-biotin (Pierce Chemical Company, Rockford, Ill. 61105; product #21330; 0.49 mM). After 3 hours (hr) of incubation at 25° C., the reaction mixture was purified by gel filtration using 10 mM NaH₂PO₄-300 mM NaCl, pH 7.00, on a Sephacryl S-200 column in 10 mM PO₄-300 mM NaCl, pH 7.00.

Procedure for biotinylation of antibody fragment via amino groups of the antibody. F(ab′)₂ solution (1 mL; 3.0 mg/mL; 30 μM) in 10 mM NaH₂PO4-300 mM NaCl, pH 7.00 was reacted with 0.0017 mL of a 10 mg/mL aqueous solution of sulfo-NHS-LC-biotin (Pierce Chemical Company, Rockford, Ill. 61105; Product #21335; 30 μM). After 3 hrs of incubation at 25° C., the reaction mixture was purified by diafiltration in an Amicon cell (YM 10 membrane) utilizing 10 mM NaH₂PO₄-300 mM NaCl, pH 7.00 buffer.

Procedure for biotinylation of reduced antibody via sulfhydryl groups of the antibody. Intact antibody (2.6 mL of 2.54 mg/mL) was mixed with 0.288 mL of dithiothreitol at 15.4 mg/mL in 100 mM NaH₂PO₄-5 mM EDTA, pH 6.0 at 37° C. for 1 hr. The reaction mixture was purified by passage through a Sephadex G25 column (1.6×45 cm) in 100 mM NaH₂PO₄-5 mM EDTA, pH 6.0 and collecting protein-containing antibody to give 6.5 mg of pure reduced antibody. The reduced antibody (6.5 mg; 43 μmole) was buffer exchanged in 10 mM NaH₂PO₄-300 mM NaCl-5 mM EDTA, pH 7.80 and later coupled with 0.023 mL (0.43 mmol) of a 10 mg/mL solution of PEO₂-iodoacetyl biotin (Pierce Chemical Company). The reaction mixture was incubated at 25° C. for 2 hr and purified over a preparative HPLC column (BioSep HPLC SEC S3000 column; 21.20×300 mm) using 10 mM NaH₂PO₄-300 mM NaCl-5 mM EDTA, pH 7.80 buffer.

Procedure for biotinylation of Fab′ fragment of an antibody via sulfhydryl groups of the antibody fragment. A solution of the F(ab′)₂ fragment (3.6 ml of 3.3 mg/mL) in 100 mM PO₄-5 mM EDTA, pH 6.0 was mixed with 0.36 mL of a solution of dithiothreitol (15.4 mg/mL in 100 mM PO₄-5 mM EDTA, pH 6.0). After heating at 37° C. for 1 hr, the protein solution was separated from excess reducing agent by passage through a Sephadex G25 column in 10 mM NaH₂PO₄-300 mM NaCl-5 mM EDTA, pH 7.80. Recovered antibody solution (3.6 mL of 2.44 mg/mL; 87.8 μM) was reacted with 0.145 mL of a 10 mg/mL aqueous solution of PEO₂-iodoacetyl biotin (Pierce Chemical Company; Product #21334; 26.7 mM). After 3 hr of incubation at ambient temperature, the reaction mixture was mixed with 0.075 mL of NEM (10 mg/mL in water; Pierce Chemical Company, product #23030). After 30 min at ambient temperature, the reaction product was purified on a Sephacryl S-200 column using 10 mM NaH₂PO₄-300 mM NaCl-5 mM EDTA, pH 7.80.

The EPRM chemibead (chemibead) was prepared in a manner similar to the method described in U.S. Pat. No. 6,153,442 and U.S. Patent Application Publication No. 20050118727A, the relevant disclosures of which are incorporated herein by reference. The EPRM chemibead comprises an aminodextran inner layer and a dexal outer layer having free aldehyde functionalities. Dexal is dextran aldehyde; see, for example, U.S. Pat. Nos. 5,929,049 and 7,172,906. The reaction is carried out at a temperature of about 0 to about 40° C., for a period of about 16 to about 64 hours at a pH of about 5.5 to about 7.0, or about 6, in a buffered aqueous medium employing a suitable buffer such as, for example, MES or the like. The reaction is quenched by addition of a suitable quenching agent such as, for example, carboxymethoxyoxime (CMO), or the like and subsequent washing of the particles. The chemiluminescent compound was 2-(4-(N,N, di-tetradecyl)-anilino-3-phenyl thioxene.

The streptavidin-sensitizer bead (sensibead) was prepared using a method analogous to that described in U.S. Pat. Nos. 6,153,442, 7,022,529, 7,229,842 and U.S. Patent Application Publication No. 20050118727A. The photosensitizer was bis-(trihexyl)-silicon-t-butyl-phthalocyanine.

The following assay formats were conducted using reagents as described above.

Assay System for Carbohydrate Antigen CA 19-9. In this Example, an embodiment of an assay method for the determination of CA 19-9, a combination was provided in a medium wherein the combination comprises (i) the sample, (ii) a photosensitizer associated with a first particle and being capable of generating singlet oxygen wherein the first particle comprises streptavidin, (iii) a chemiluminescent composition activatable by the singlet oxygen and associated with a second particle wherein the second particle comprises anti-CA 19-9 antibody (chemibead reagent) and (iv) a conjugate of an antibody for CA 19-9 and biotin prepared as described above (anti-CA19.9 antibody from Fujirebio Diagnostics, Inc., Malvern, Pa.). The combination was subjected to conditions for binding of CA 19-9, if present, to the antibody for CA 19-9. The reaction mixture was combined with the first particle containing streptavidin. The photosensitizer particle was irradiated with light and the amount of luminescence generated by the chemiluminescent composition is detected, the amount of luminescence being related to the presence and/or amount of CA 19-9 in the sample.

The following reagents were utilized in the above assay system:

Generic Diluent. Final buffered solution was formulated to contain 57.5 mM HEPES-300 mM NaCl-1.15 mM EDTA-1.0% dextran T-500-0.1% TRITON® X-405 surfactant-0.15% PROCLIN® 300-0.01% neomycin sulfate, pH 8.0.

Chemibead Reagent. The chemibeads were diluted to a concentration of 100 μg/mL in generic diluent.

Antibody Reagent. Biotinylated anti-CA 19-9 monoclonal antibodies (prepared as described above) were diluted to a concentration of 10 μg/mL in generic diluent containing 1 mg/mL mouse IgG, 1 mg/mL bovine serum albumin and 1 mg/mL bovine gammaglobulin.

Sensibead Reagent. Sensibeads were diluted to 1.5 mg/mL in generic diluent.

As mentioned above, assays were carried out on a DIMENSION VISTA® analyzer using a sample solution containing carbohydrate antigen CA 19-9. At time t=zero sec., 20 μL biotinylated antibody reagent, 20 μL chemibead reagent and 15 μL water were added to a reaction vessel. Sample, 10 μL, was added 21.6 seconds later, followed by 15 μL water. After 291.6 seconds, 20 μL sensibead reagent was dispensed. Measurements were taken 601.2 seconds after initiation of the reaction sequence.

The procedure described above was applied to multiple sample solutions having known CA 19-9 concentrations using various biotinylated reagents as discussed herein. For each solution, read values correlating to a known concentration were plotted (counts or kilocounts as a function of known concentration).

Variation of number of biotin molecules in the biotin reagent with alkylene linker. The moles of biotin incorporated, controlled by molar challenge ratio of the biotin reagent, was studied in these experiments. Biotinylated antibody, prepared with equimolar challenge amount of the biotin reagent (NHS-LC-biotin) showed an increase in signal separation by a factor of two (L6-L1, 2630 kcounts; experiment (expt) 1 Table 1) compared to those prepared by 2 or 5 fold molar excess of the reagent (L6-L1, 1107 or 1569 kcounts; experiments 2 and 3, respectively, Table 1). These results demonstrate that the number of biotins incorporated can be utilized in controlling performance of the biotinylated antibody. Variations in this feature allow one to choose a biotin challenge ratio to produce a biotin-antibody reagent that provides optimal sensitivity for an analyte determination in an assay. Ratio L2/L1 represents low end of the calibration curve and is an indication of the assay sensitivity. L6/L1 and L6-L1 represent ratio and the differences in signals, respectively, generated at the highest and the lowest analyte concentration and represent total calibration curve.

TABLE 1
Dose-dependent response (kcounts) with biotin reagents
prepared from F(ab′)₂ reacted with
NHS-LC-biotin at various molar challenge ratios

| | Response (kcounts) | | |
|---|---|---|---|
| | Expt 1 | Expt 2 | Expt 3 |
| Analyte conc. | F(ab′)₂- | F(ab′)₂- | F(ab′)₂- |
| (μg/mL) | LC-Biotin (1:1) | LC-Biotin (2:1) | LC-Biotin (5:1) |
| 0.0 | 32 | 14 | 17 |
| 30.0 | 106 | 55 | 65 |
| 131.0 | 396 | 201 | 242 |
| 263.0 | 843 | 394 | 495 |
| 525.0 | 1863 | 775 | 1033 |
| 1050.0 | 2662 | 1121 | 1586 |
| L2/L1 | 3.30 | 3.86 | 3.78 |
| L6/L1 | 83.2 | 78.7 | 91.7 |
| L6-L1 | 2630 | 1107 | 1569 |

Variation of the length of the spacer group in the biotin reagent. Experiments were conducted in which the length of an aliphatic chain present in the biotin reagent is varied. The biotin reagents employed in spacer to the antibody were a C₆-alkyl-derived chain (-LC-biotin; experiment 1, Table 2) and a C₁₋₂-alkyl-derived chain (-LC-LC-biotin; experiment 2, Table 2). The results indicated that the presence of an additional six carbons (C₆ versus C₁₂) in the alkyl-derived chain decreased performance of the corresponding biotinylated antibody by a factor of three. Variations in this feature allow one to choose a biotin reagent that provides optimal sensitivity for an analyte.

TABLE 2
Dose-dependent response (kcounts) with biotin
reagents prepared from F(ab′)₂ reacted with
biotin derivatives containing different spacer arms

| | |
|---|---|
| Analyte | Response (kcounts) |

| conc. | Expt 1 | Expt 2 |
|---|---|---|
| (μg/mL) | F(ab′)₂ LC-Biotin (5:1) | F(ab′)₂-LCLC-Biotin (5:1) |
| 0.0 | 17 | 9 |
| 30.0 | 65 | 27 |
| 131.0 | 242 | 88 |
| 263.0 | 495 | 172 |
| 525.0 | 1033 | 333 |
| 1050.0 | 1586 | 517 |
| L2/L1 | 3.78 | 2.88 |
| L6/L1 | 91.7 | 55.2 |
| L6-L1 | 1569 | 508 |

Variation of number of biotin molecules in the biotin reagent with (PEO)₄ spacer group. The moles of biotin incorporated, controlled by molar challenge ratio of the biotin reagent, was studied in these experiments. Biotinylated antibody, prepared with equimolar challenge amount of the biotin reagent (NHS-(PEO)₄-biotin) showed an increase in signal separation by a factor of ten (L6-L1 337; experiment 1, Table 3) compared to those prepared by 10 or 30 fold molar excess of the reagent (L6-L1 35 or 38; experiments 2 and 3, respectively, Table 3). These results further demonstrate that the number of biotins incorporated and the nature of the spacer group can be utilized in controlling performance of the biotinylated antibody in an assay. Variations in this feature allow one to choose a biotin reagent that provides optimal sensitivity for an analyte, which includes an increased or decreased sensitivity or dynamic range.

TABLE 3
Dose-dependent response (kcounts) with
biotinylated-F(ab′)₂ reagents prepared by reaction
with NHS-(PEO)₄-biotin at various molar challenge ratios

| | Response (kcounts) | | |
|---|---|---|---|
| Analyte | Expt 1 | Expt 2 | Expt 3 |
| conc. | F(ab′)₂ | F(ab′)₂ | F(ab′)₂ |
| (μg/mL) | PEO₄-Biotin (5:1) | PEO₄-Biotin (10:1) | PEO₄-Biotin (30:1) |
| 0.0 | 17 | 8 | 7 |
| 30.0 | 35 | 10 | 10 |
| 131.0 | 95 | 17 | 21 |
| 263.0 | 161 | 25 | 31 |
| 525.0 | 259 | 36 | 41 |
| 1050.0 | 354 | 43 | 45 |
| L2/L1 | 2.09 | 1.27 | 1.43 |
| L6/L1 | 20.9 | 5.5 | 6.4 |
| L6-L1 | 337 | 35 | 38 |

Comparison of biotinylated IgG and biotinylated Fab′. Biotinylated IgG (L6-L1 4849, expt 1, Table 4) exhibited increased signal separation when compared to the biotinylated Fab′ (L6-L1 3216, expt. 2; Table 4), where free sulfhydryls of the protein were used to react with iodoacetyl-(PEO)₂-biotin. In both reagents, the incorporated biotins are located away from the antigen-binding site. Reduction of IgG generated an average of 10-16 free sulfhydryls in the hinge region of the protein compared to about 4 free sulfhydryls present at the C-terminal of the Fab′ fragment.

TABLE 4
Dose-dependent response (kcounts) with
biotin reagents prepared by reaction of the
antibody with free sulfhydryl-directed biotin derivatives

Response (kcounts)

| Analyte | Expt 1 | Expt 2 |
|---|---|---|
| conc. | IgG-acetyl-PEO₂- | F(ab′)-acetyl- |
| (μg/mL | Biotin (10:1) | PEO₂-Biotin (10:1) |
| 0.0 | 95 | 32 |
| 30.0 | 283 | 111 |
| 131.0 | 1011 | 440 |
| 263.0 | 2066 | 992 |
| 525.0 | 3764 | 2141 |
| 1050.0 | 4944 | 3248 |
| L2/L1 | 2.97 | 3.51 |
| L6/L1 | 51.9 | 102.7 |
| L6-L1 | 4849 | 3216 |

A biotinylated IgG, prepared by the supplier (Fujirebio Diagnostics, Inc.) using IgG and NHS-LC-biotin from Pierce Chemical Company according to the manufacturer's (Pierce Chemical Company) instructions that included a carbonate buffer (pH≧8.0), was evaluated in the above assay system and the results are summarized in Table 5 (Expt 1, Table 5). The designation (5:1) in Table 5 refers to a 5-fold molar excess of NHS-LC-biotin reagent over IgG.

TABLE 5
Dose-dependent response (kcounts) of the
biotin reagent prepared by reaction of the
antibody with five fold molar excess of NHS-LC-biotin

| Analyte | Response (kcounts) |
|---|---|
| conc. | Expt 1 |
| (μg/mL) | IgG LC-Biotin (5:1) |
| 0.0 | 52 |
| 30.0 | 180 |
| 131.0 | 702 |
| 263.0 | 1533 |
| 525.0 | 3002 |
| 1050.0 | 4110 |
| L2/L1 | 3.45 |
| L6/L1 | 78.8 |
| L6-L1 | 4057 |

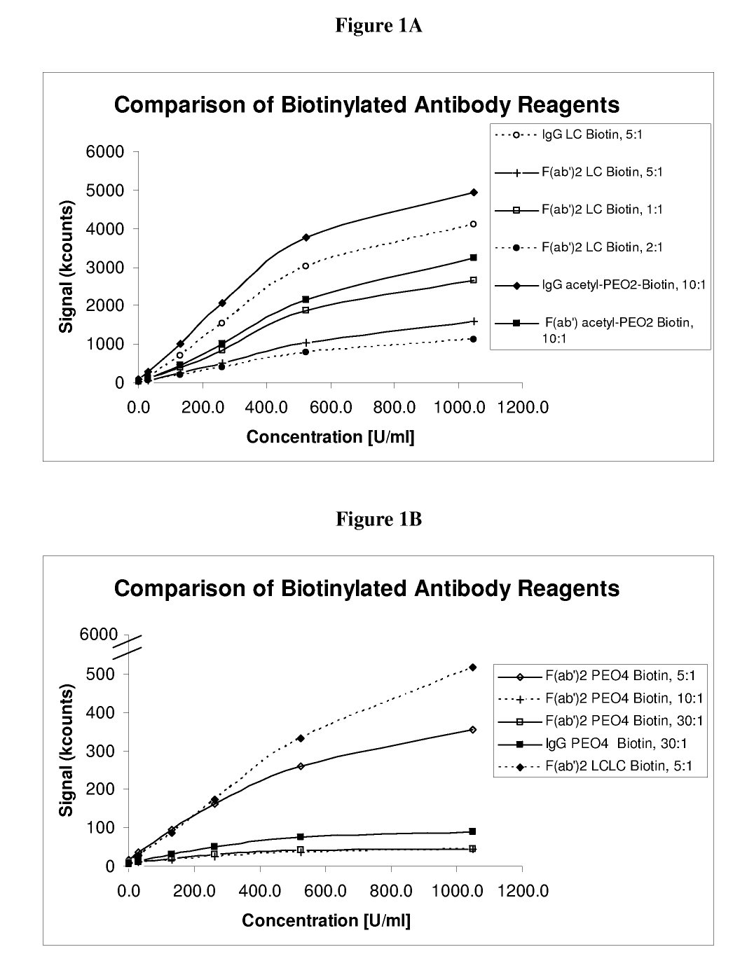

The results of the above experiments are summarized in FIGS. 1A and 1B, which show modulation of the signal response of the biotinylated reagents in order of decreasing signal separation. A logit curve fit calculation was used to generate a standard curve. As can be seen, the nature of the spacer group and the number of biotin molecules in the biotinylated reagent has a significant impact on the signal curves versus concentration of analyte. Thus, in accordance with the modulation concept of the present invention, one can choose a suitable biotinylated reagent for the assay system (including the detector) in question so that an amount of signal produced will be optimized for such assay system. As mentioned above, a number of factors are involved in achieving an optimized sensitivity. Such factors include the nature of the measuring or detecting system, the range of signal detection of the detecting system, saturation of the detection system due to extent of signal generation, a large variation in the analyte concentration present in the sample to be analyzed and the like. Thus, for a particular set of conditions for the assay system, one of the biotinylated reagents in FIG. 1A or 1B might be preferred over another based on the above factors. For instance, a biotinylated reagent that achieves a maximum amount of signal may not be preferred over another biotinylated reagent that achieves a lesser amount of signal but is preferred because of, for example, the range of signal detection of a detector.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

What is claimed is:

1. A method for designing an antibody reagent for use in an assay for the detection of an analyte to obtain an optimum assay sensitivity wherein the antibody reagent is a conjugate of a small molecule attached by a spacer group to an antibody for the analyte, the method comprising controlling, in the preparation of the conjugate, reaction parameters comprising the hydrophobicity or hydrophilicity of the spacer group, the length of the spacer group, the number of molecules of the small molecule attached to the antibody and the point of attachment of the small molecule to the antibody to obtain an optimum assay sensitivity.

2. The method according to claim 1 comprising:
(a) preparing two or more conjugates by selecting a set of parameters for each conjugate wherein the set of parameters is different for each conjugate,
(b) conducting an assay for the analyte employing each conjugate and
(c) selecting for use in the assay the conjugate that provides the optimum assay sensitivity.

3. The method of claim 1 wherein the small molecule is attached to amino groups of intact antibody or a fragment thereof or sulfhydryl groups in the hinge region of intact antibody or a fragment thereof by means of a spacer group that comprises a chain of about 2 to about 18 atoms in length wherein the chain comprises carbon or comprises carbon and at least one heteroatom with the proviso that when the antibody is a Fab′ fragment, the small molecule is linked to the antibody by means of a spacer group that comprises carbon and at least one heteroatom or the small molecule is linked to the antibody by means of one or more amino groups of the antibody.

4. The method according to claim 1 wherein the antibody of the conjugate is intact IgG and wherein the small molecule is linked to the antibody by means of one or more amino groups of the IgG.

5. The method according to claim 1 wherein the antibody of the conjugate is an antibody fragment and wherein the small molecule is linked to the antibody by means of one or more amino groups of the antibody fragment.

6. The method according to claim 1 wherein the antibody of the conjugate is intact IgG or a Fab′ fragment and wherein the small molecule is linked to the antibody by means of a spacer group to one or more sulfhydryls produced in the intact antibody or the Fab′ fragment wherein the spacer group comprises carbon and at least one heteroatom.

7. The method according to claim 1 wherein the spacer group has the formula:
—CH₂(CH₂)ₙCH₂— wherein n is 4 to 7 or (I)
—CH₂(CH₂CH₂O)ₘ— wherein m is 2 to 4. (II)

8. The method according to claim 1 wherein the small molecule is biotin.

9. The method according to claim 1 further comprising controlling the number of molecules of the small molecule in the conjugate by controlling the molar challenge ratio of a small molecule-derivatizing agent to the antibody or the fragment thereof in the preparation of the conjugate.

10. A method for determining the presence and/or amount of an analyte in a sample suspected of containing the analyte, the method comprising:
(a) providing in combination in a medium:
(i) the sample,
(ii) an antibody reagent prepared according to the method of claim 1 wherein the antibody reagent comprises an antibody for the analyte,
(iii) a binding moiety for the small molecule wherein the binding moiety is conjugated to a support, a member of a specific binding pair or a member of a signal producing system, and
(iv) an analyte analog or a second antibody for the analyte,
(b) subjecting the combination to conditions for binding of the analyte to the antibody reagent comprising an antibody for the analyte, and
(c) determining the extent of binding of the analyte to the antibody reagent, the extent of the binding being related to the presence and/or amount of the analyte in the sample.

11. A method for designing a biotinylated antibody reagent for use in an assay for the detection of an analyte to obtain an optimum assay sensitivity wherein the biotinylated antibody reagent is a conjugate of biotin attached by a spacer group to an antibody for the analyte, the method comprising controlling, in the preparation of the conjugate, reaction parameters comprising:
(a) the hydrophobicity or hydrophilicity of the spacer group,
(b) the length of the spacer group wherein the spacer group comprises a chain of about 2 to about 18 atoms in length wherein the chain comprises carbon or comprises carbon and at least one heteroatom,
(c) the number of molecules of biotin attached to the antibody wherein the number of molecules of biotin in the conjugate is controlled by controlling the molar challenge ratio of a biotin-derivatizing agent to the antibody or the fragment thereof in the preparation of the conjugate and
(d) the point of attachment of biotin to the antibody wherein the biotin is attached to amino groups of intact antibody or a fragment thereof or sulfhydryl groups in the hinge region of intact antibody or a fragment.

12. The method according to claim 11 comprising:
(a) preparing two or more conjugates by selecting a set of parameters for each conjugate wherein the set of parameters is different for each conjugate,
(c) conducting an assay for the analyte employing each conjugate and
(c) selecting for use in the assay the conjugate that provides the optimum assay sensitivity.

13. The method of claim 11 wherein, when the antibody is a Fab′ fragment, biotin is linked to the antibody by means of a spacer group that comprises carbon and at least one heteroatom or biotin is linked to the antibody by means of one or more amino groups of the antibody.

14. The method according to claim 11 wherein the antibody of the conjugate is intact IgG and wherein the small molecule is linked to the antibody by means of one or more amino groups of the IgG.

15. The method according to claim 11 wherein the antibody of the conjugate is an antibody fragment and wherein the small molecule is linked to the antibody by means of one or more amino groups of the antibody fragment.

16. The method according to claim 11 wherein the antibody of the conjugate is intact IgG or a Fab′ fragment and wherein the small molecule is linked to the antibody by means of a spacer group to one or more sulfhydryls produced in the intact antibody or the Fab′ fragment wherein the spacer group comprises carbon and at least one heteroatom.

17. The method according to claim 11 wherein the spacer group has the formula:
—CH₂(CH₂)ₙCH₂— wherein n is 4 to 7 or (I)
—CH₂(CH₂CH₂O)ₘ— wherein m is 2 to 4. (II)

18. A method according to claim 11 wherein the assay further comprises other reagents for determining the presence and/or amount of an analyte in a sample suspected of containing the analyte, the reagents comprising:
(a) a binding moiety for the small molecule wherein the binding moiety is conjugated to a support, a member of a specific binding pair or a member of a signal producing system, and
(b) an analyte analog or a second antibody for the analyte.

19. A method for determining the presence and/or amount of an analyte in a sample suspected of containing the analyte, the method comprising:
(a) providing in combination in a medium:
(i) the sample,
(ii) a biotinylated antibody reagent prepared by the method of claim 1 wherein the biotinylated antibody reagent comprises an antibody for the analyte,
(iii) a biotin-binding moiety wherein the biotin-binding moiety is conjugated to a support, a member of a specific binding pair or a member of a signal producing system, and
(iv) an analyte analog or a second antibody for the analyte,
(b) subjecting the combination to conditions for binding of the analyte to the antibody reagent comprising an antibody for the analyte, and
(c) determining the extent of binding of the analyte to the antibody reagent, the extent of the binding being related to the presence and/or amount of the analyte in the sample.
